(19) United States
(12) Patent Application Publication
(10) Pub. No.: US 2008/0281105 A1
(43) Pub. Date: Nov. 13, 2008

(54) Novel Quinolinium Salts and Derivatives

(75) Inventors: James E. Macdonald, Encinitas, CA (US); Michelle K. Hysell, San Diego, CA (US); Dehua Yu, San Diego, CA (US); Henry Li, Carlsbad, CA (US); Flossie Wong-Staal, San Diego, CA (US)

Correspondence Address: LAW OFFICE OF DAVID SPOLTER;c/o G. Singer, 30 South Adelaide Avenue, Suite #5M, Highland Park, NJ, 08904, US

(73) Assignee: IMMUSOL INCORPORATED, San Diego, CA (US)

(21) Appl. No.: 11/814,036

(22) PCT Filed: Jan. 18, 2006
(86) PCT No.: PCT/US2006/001793

§ 371 Date: Jun. 2, 2008

Related U.S. Application Data
(60) Provisional application No. 60/645,093, filed on Jan. 18, 2005.
(60) Provisional application No. 60/715,257, filed on Sep. 8, 2005.

(51) Int. Cl. C07D 215/38; C07D 401/06

(52) U.S. Cl. 546/171; 546/176

(57) ABSTRACT

The present invention relates generally to the synthesis of novel quinolinium salts and derivative compounds. Such salts and compounds are useful for inhibiting the growth of cancer cells.

### FIELD OF THE INVENTION

The present invention relates generally to the synthesis of novel salts and derivative compounds. Such salts and compounds can inhibit the growth of cancer cells. In particular, the invention provides novel quinolinium salts as well as novel quinolinium derivative compounds.

### BACKGROUND INFORMATION

Several patents provide for the preparation of 1-alkyl-2-(2-substituted-vinyl) quinolinium salts, give physical properties, and uses. Brooker describes the preparation of (1-methyl-2-quinoline)-1-laural-2,5-dimethyl-3-pyrrole)dimethinecyanine p-toluenesulfonate and use as photographic agents.¹ Additional 2-vinylquinoliniums are described as photographic agents, including 6-dimethylamino-1-methyl-2-[(1-phenyl-4-pyrazoyl)vinyl]quinolinium iodide, 6-dimethylamino-1-methyl-2-[(3,5-dimethyl-1-phenyl-4-pyrazoyl)vinyl]quinolinium iodide, and 1-ethyl-2-[(3,5-dimethyl-1-phenyl-4-pyrazoyl)vinyl]-6-nitroquinolinium iodide² and the 1-methyl-2-2′-3″pyridylvinylquinolinium iodide.³ A few of such compounds have been described as being effective against worm infestation and are listed in Table 1.⁴

TABLE 1
Quinolinium nematocide compounds

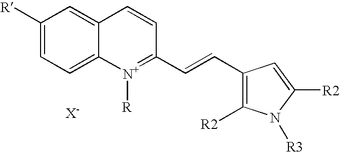

| R | R′ | R2 | R3 | X |
|---|---|---|---|---|
| Me | Me | Me | Ph | Toluenesul- |
| | | | | fonate |
| Me | Me | Me | Et | Iodide |
| Me | Me | Me | Et | Chloride |
| Me | Me | Me | Laural(Dodecyl) | Iodide |
| Me | Me | Me | Laural(Dodecyl) | Chloride |
| Et | Me | Me | Ph | Chloride |
| Me | Me | Me | Heptyl | Chloride |
| Me | Me | Me | Me | Chloride |
| Me | Me | Me | Hexadecyl | Chloride |
| Heptyl | Me | Me | Ph | Chloride |
| Dodecyl | Me | Me | Ph | Chloride |
| 2-ethoxyethyl | Me | Me | Ph | Chloride |
| Me | Me | Me | CH2Ph | Iodide |
| Me | Me | Me | Amyl | Iodide |
| Me | Me | Me | Amyl | chloride |
| 2-hydroxyethyl | Me | Me | Ph | Iodide |
| 2-hydroxyethyl | Me | Me | Ph | chloride |
| Me | Me | Me | 2-methoxyethyl | Iodide |
| Me | Me | Me | 2-methoxyethyl | Chloride |
| Me | dimethylamino | Me | Ph | Iodide |
| Me | dimethylamino | Me | Ph | Chloride |
| Me | Me | Me | 4-ClPh | Chloride |
| Me | Me | Me | 4-EtOPh | Chloride |
| Amyl | Me | Me | Amyl | Iodide |
| Me | Me | Me | Butyl | Iodide |
| Me | Me | Me | Decyl | Iodide |
| Me | Me | Me | Isopropyl | Iodide |
| Me | Cl | Me | Ph | iodide |
| Me | Cl | Me | Ph | Chloride |
| Et | Me | Me | Amyl | Iodide |
| Et | Me | Me | Amyl | chloride |
| Propyl | Me | Me | Amyl | Iodide |
| Propyl | Me | Me | Amyl | chloride |
| Me | Me | Me | Cyclohexyl | Iodide |
| Me | Me | Me | Cyclohexyl | chloride |
| Me | Me | Me | Propyl | Iodide |
| Me | Me | Me | Propyl | chloride |
| Me | MeO | Me | Amyl | Chloride |
| Me | MeO | Me | Amyl | Iodide |
| Me | Enanthamino | Me | Ph | Iodide |
| Me | Enanthamino | Me | Ph | Iodide |
| Me | Ph | Me | Ph | Iodide |
| Me | Ph | Me | Ph | chloride |

The chemistry literature also has references to the preparation of 1-alkyl-2-(2-ary or 2-heteroaryl substituted-vinyl)quinolinium salts, and their physical and spectral properties. For example, Wizinger reported on the preparation of N-methyl-2-[2-[2-hydroxynaphthyl-1-yl]vinyl]quinolinium perchlorate as orange needles, mp 266° C. and its color change with base.⁵

Lugowkin prepared 1-Methyl-2-[(E)-2-(1,3,7-trimethyl-2,6-dioxo-2,3,6,7-tetrahydro-1H-purin-8-yl)-vinyl]-quinolinium and 1-Ethyl-2-[(E)-2-(1,3,7-trimethyl-2,6-dioxo-2,3,6,7-tetrahydro-1H-purin-8-yl)-vinyl]-quinolinium iodides from quinolinium and caffenine-8-aldehyde in acetic anhydride and reported physical properties of the dyes.⁶

Lugowkin also prepared 1-Methyl-2-[(E)-2-(1,3,9-trimethyl-2,6-dioxo-2,3,6,9-tetrahydro-1H-purin-8-yl)-vinyl]-quinolinium and 1-Ethyl-2-[(E)-2-(1,3,9-trimethyl-2,6-dioxo-2,3,6,9-tetrahydro-1H-purin-8-yl)-vinyl]-quinolinium iodides from quinolinium and isocaffenine-8-aldehyde in acetic anhydride, and reported physical properties of the new compounds.⁷

Zhmurova reported the preparation, physical properties and dye absorption maxima of 2-[(E)-2-(phenyl)-vinyl]-1-ethyl-quinolinium perchlorate, 2-[(E)-2-(4-Methyl-phenyl)-vinyl]-1-ethyl-quinolinium perchlorate, 2-[(E)-2-(4-hydroxy-phenyl)-vinyl]-1-ethyl-quinolinium perchlorate, 2-[(E)-2-(4-amino-phenyl)-vinyl]-1-ethyl-quinolinium perchlorate, 2-[(E)-2-(4-(Dimethylamino)-phenyl)-vinyl]-1-ethyl-quinolinium perchlorate and 2-[(E)-2-(4-(triphenyphosphoranylidene-phenyl)-vinyl]-1-ethyl-quinolinium perchlorate.⁸

Pilyugin reported the preparation, physical properties and adsorption spectra of the dye acid salts and free bases of 2-[(E)-2-(2-Hydroxy-phenyl)-vinyl]-1-methyl-quinolinium perchlorate, 2-[(E)-2-(2-Hydroxy-phenyl)-vinyl]-1-phenyl-quinolinium perchlorate, 1-decyl-2-[(E)-2-(2-Hydroxy-phenyl)-vinyl]-quinoliniumperchlorate, 1-decyl-2-[(E)-2-(2-Hydroxy-phenyl)-vinyl]-6-methoxy-quinolinium perchlorate, 2-[(E)-2-(2-Hydroxy-phenyl)-vinyl]-1-methyl-quinolinium perchlorate, 2-[(E)-2-(2-Hydroxy-3-nitro-phenyl)-vinyl]-1-phenyl-quinoliniumperchlorate, 1-decyl-2-[(E)-2-(2-Hydroxy-3-nitro-phenyl)-vinyl]-quinoliniumperchlorate, 1-decyl-2-[(E)-2-(2-Hydroxy-3-nitro-phenyl)-vinyl]-6-methoxy-quinolinium perchlorate, 2-[(E)-2-(2-Hydroxy-1-naphthyl)-vinyl]-1-methyl-quinolinium perchlorate, 2-[(E)-2-(2-Hydroxy-1-naphthyl)-vinyl]-1-phenyl-quinoliniumperchlorate, 1-decyl-2-[(E)-2-(2-Hydroxy-1-naphthyl)-vinyl]-quinolinium perchlorate and 1-decyl-2-[(E)-2-(2-Hydroxy-1-naphthyl)-vinyl]-6-methoxy-quinolinium perchlorate.⁹

Stepanov reported the preparation and UV spectra for 2-[(E)-2-(2,3-Dimethyl-indolizin-1-yl)-vinyl]-1-methyl-quinolinium perchlorate, 2-[(E)-2-(2,3-Dimethyl-indolizin-1-yl)-vinyl]-1-ethyl-quinolinium perchlorate, and 2-[(E)-2-(2,3-Dimethyl-indolizin-3-yl)-vinyl]-1-methyl-quinolinium perchlorate.¹⁰

Chernyuk reported the preparation of 2-[[4-(dimethylamino)phenyl]vinyl]-1-methylquinolinium perchlorate by the use of 2-(triphenylphosphonium)methyl-1-methylquinolinium diperchlorate.¹¹ Buttgereit and Scheibe reported on the spectral shift on reaction with pyrrolidine of 2-[[4-(dimethylamino)phenyl]vinyl]-1-methylquinolinium perchlorate.¹²

Mal'tseva reported the preparation, mp elemental analysis, and spectral absorption maxima of the 2-[(E)-2-(1,3-Dimethyl-1H-pyrazol-4-yl)-vinyl]-1-methyl-quinolinium perchlorate and 2-[(E)-2-(1,5-Dimethyl-1H-pyrazol-4-yl)-vinyl]-1-methyl-quinolinium perchlorate.¹³

Rout prepared and measured the optical properties of pyrazolone quinoline dimethine dyes.¹⁴

Boyer reported the spectral shifts of cyanine and merocyanine dyes in solution and AgBr pellets.¹⁵

Osman reported the preparation and adsorption spectra of 2-((E)-2-Benzooxazol-2-yl-vinyl)-1-ethyl-quinolinium iodide and 2-((E)-3-ethyl-2-Benzooxazolium-2-yl-vinyl)-1-ethyl-quinolinium diiodide.¹⁶ Wainright reported the weak antibacterial activity of 2-(4-dimethylaminostryrl)-1-methylquinoliniums substituted in the quinoline 6 position with hydrogen, methoxy, amino and hexylamide.¹⁷ Malina reported that the 6-(aminocarbonyloxy)-2-(2-(8-hydroxy-5-methylquinolin-7-yl)vinyl)-1-methylquinolinium (NSC 86372) inhibited protein synthesis in a eukaryotic translation system.

Pyrvinium is a compound with a similar structure as those discussed above. Specifically, it has the following structure:

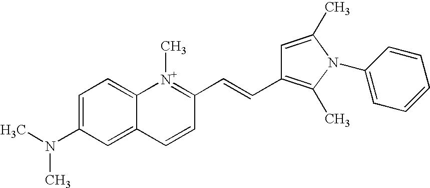

It has been shown to have antitumor activity. More specifically, its pamoate salt has been shown by Esumi to have preferential toxicity for various cancer cell lines during glucose starvation.¹⁸ In addition, the pyrvinium pamoate was orally dosed as a suspension in 2% DMSO in water to nude mice with a pancreatic tumor. While somewhat effective, this formulation demonstrated a saturation of response, giving the same results at 100 and 200 ug/mouse due to limited solubility.

In a broader sense, the current forms of pyrvinium are not optimum for the treatment of cancer. For example, the pyrvinium chloride salt has an intensely bitter taste.¹⁹ In addition, the iodide form is not a preferred pharmaceutical salt and can lead to symptoms such as skin rash and headache and even toxicity.²⁰ Additional unsuitable salt forms of pyrvinium include the methosulfate,²¹ and Phenolphthalein.²² Sodium methyl sulfate has been reported to be a mutagen.²³ Phenolphthalein is listed as a carcinogen by California.²⁴

Finally, as discussed above, the limited solubility of the pamoate form gives dose limiting effects. A need therefore exists to develop better salt forms of pyrvinium, as well as novel pyrvinium derivative compounds.

Another quinolinium derivative compound, quinaldine red (depicted below), has been claimed to have anticancer activity. More specifically, it has been reported as an inhibitor of transformed cell growth.²⁵

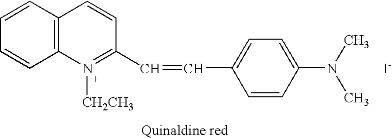

Thus, there is also a need to make additional novel quinolinium derivative compounds, which also have anti-tumor activity. The subject invention satisfies these needs and provides related advantages as well.

### SUMMARY OF THE INVENTION

The present invention provides novel salt forms of the compound pyrvinium, which is known to have anti-tumor activity. According to a first aspect, the invention provides salt forms of the compound with improved aqueous solubility. According to another aspect, the invention provides forms of the compound that incorporate acidic compounds with anti-tumor activity. In yet a further aspect, the invention provides forms of pyrvinium with an acid ion exchange polymer, which is useful for oral dosing. In an additional aspect, a compound of the invention is combined with another chemotherapeutic agent.

The present invention further provides novel quinolinium compound derivatives with the following formula:

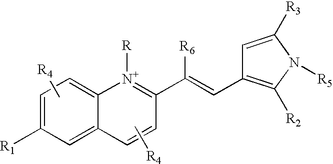

with the meaning of the variables described below.

The present invention further provides novel quinolinium compound derivatives with the following formula:

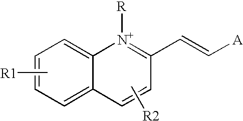

with the meaning of the variables described below.

### BRIEF DESCRIPTION OF THE DRAWINGS

FIG. 1 shows reduction in tumor growth of colon cancer cells (as compared to control) using the compound of Example 1.

FIG. 2 shows reduction in tumor growth of non-small lung cancer cells (as compared to control) using the compound of Example 56.

FIG. 3 shows the effects of the combination therapy of the compound of Example 1 and doxorubicin for PC3 medium-staged xenograft tumors.

FIG. 4 shows the effects of the combination therapy of the compound of Example 1 and taxol for PC3 medium-staged xenograft tumors.

FIG. 5 shows the anti-tumor activity of the compound of Example 47 combined with taxol.

FIG. 6 shows the anti-tumor activity of the compounds of Example 36 and Example 47 in a Lewis lung cancer xenograft model.

FIG. 7 shows the anti-tumor activity of the combination of the compound of Example 36 with 2-deoxyglucose (2DG) in a NSCL xenograft model.

FIG. 8 shows anti-tumor activity of the combination of the compound of Example 36 with 2DG in a Lewis lung cancer xenograft model.

### DETAILED DESCRIPTION OF THE INVENTION

The present invention relates to novel salts of the compound pyrvinium, which has the chemical name of 6-(dimethylamino)-2-[2-(2,5-dimethyl-1-phenyl-1H-pyrrol-3-yl)ethenyl]-1-methyl-quinolinium and the following structure:

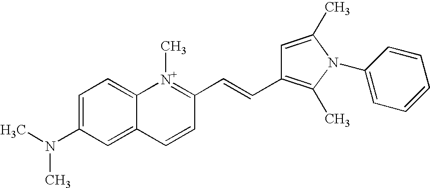

More specifically, the invention provides salts of this compound that improve its water solubility. Accordingly, salt forms of pyrvinium include the following: acetate, trifluoroacetate, carbonate, bicarbonate, benzoate, salicylate, glucuronate, lactate, tartrate, mucate, gluconate, succinate, glutamate, aspartate, maleate, citrate, glutarate, phosphate, sulfate, methanesulfonate, trifluoromethanesulfonate, tosylate, benzenesulfonate, 1,2-ethanedisulfonate, 2-hydroxyethanesulfonate, 2-naphthalenesulfonate, ethanesulfonate, camphorsulfonate, sulfamate and cyclohexylsulfamate. Preferred salt forms are sulfate and phosphate. See Examples 1 and 2.

The present invention also provides novel salt forms of pyrvinium that incorporate acidic compounds with additional useful anticancer activity. Examples of such salts include, but are not limited to, 2-Mercaptopyridine-N-oxide [CAS Reg. No. 1121-31-9], Ciclopirox [CAS Reg. No. 29342-05-0], acetyl-11-keto-beta-boswellic acid, celastrol, dihydrocelastrol, Glycyrrhizic acid [CAS Reg. No. 53956-04-0], Ursolic acid [CAS Reg. No. 77-52-1] and 18-β-Glycyrrhetinic acid, [CAS Reg. No. 471-53-4]. Moreover, the present invention provides a novel form of pyrvinium useful for oral dosing. Such form of pyrvinium salt is prepared using an ion exchange polymer such as a strong acid polymer resin to give a stable salt form, masking the bitter taste of pyrvinium, and providing a controlled or sustained release of the pyrvinium cation. See, for example: http://wwwdotrohmhaas.com/ionexchange/Pharmaceuticals/Formulations_doc/us_english/Irp69 dotPDF.

An example of such a resin is the sulfonated polystyrene Amberlite IRP-69. Alternatively, the strong acid polymer resins Dowex-50×8 or Dowex-50×2 may also be used to make new salt forms of pyrvinium. Other acidic non-polystyrene polymer resins may also be used, such as acrylate or acrylamide polymers. In addition, Amberlite IRP69, has been used to provide controlled or sustained release of dextromethorphan.

The new salt forms described above can be prepared by treating pyrvinium pamoate with the acid form of the new salt in a solvent. The precipitate of the new salt form can then be collected.

Alternatively, the new salt forms can be prepared by treating pyrvinium chloride or iodide with the silver form of the new salt in a solvent, and removing the precipitated silver halide.

Alternatively, the new salt forms can be prepared by treating pyrvinium chloride with the hydrogen, sodium or potassium form of the new salt in a solvent, and collecting the new salt form.

Alternatively, pyrvinium can be exchanged with a strong acid ion exchange resin in its hydrogen, ammonium, sodium or potassium form, to provide a resin bound pyrvinium. This may be used as is, or the pyrvinium may be subsequently removed from the resin with by treatment with a solution of another acid in its hydrogen, ammonium sodium or potassium form.

The present invention also relates to compounds of the following formula:

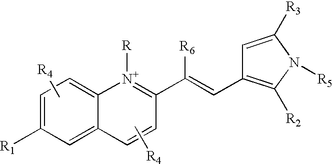

wherein:

R is C₁ to C₁₂ alkyl, C₂ to C₁₂ alkenyl, C₂ to C₁₂ alkynyl, C₁ to C₁₂ substituted alkyl,
C₂ to C₁₂ substituted alkenyl, C₂ to C₁₂ substituted alkynyl, C₇ to C₁₈ phenylalkyl or
C₇ to C₁₈ substituted phenylalkyl;
or R and R₆ may form a heterocycle or substituted heterocycle;
or R and R₄ may form a heterocycle or substituted heterocycle;
R₁ is hydrogen, alkyl, alkoxy, halo, cyano, nitro, CO₂R₇, C(O)NR₇R₈, —CH═NR₇,
C═NR₉—NR₇R₈, or —NR₇R₈, wherein R₇ and R₈ are, independently, C₁ to C₁₂ alkyl, C₂ to C₁₂ alkenyl, C₂ to C₁₂ alkynyl, C₁ to C₁₂ substituted alkyl, C₂ to C₁₂ substituted alkenyl, C₂ to C₁₂ substituted alkynyl, C₇ to C₁₈ phenylalkyl or C₇ to C₁₈ substituted phenylalkyl; or —NR₇R₈ is heterocycle, substituted heterocycle, cyclic C₂ to C₇ heteroalkylene or substituted cyclic C₂ to C₇ heteroalkylene, wherein the substituted heterocycle and substituted cyclic C₂ to C₇ heteroalkylene are defined below and also include oxo and a substitution of the formula ═NR₉, wherein R₉ is hydrogen, C₁ to C₁₂ alkyl or C₁ to C₁₂ substituted alkyl;
R₂, R₃, R₄ and R₆ are, independently, hydrogen, C₁ to C₁₂ alkyl, C₁ to C₁₂ substituted alkyl, halo or one of the following the formulae: a) —(CH₂)ₙCO₂R₁₀;
b) —CH₂)ₙCON(R₁₀)₂; c) —(CH₂)ₙCN; or d) (CH₂)ₙSO₂R₁₀; wherein R₁₀ is hydrogen, C₁ to C₁₂ alkyl or C₁ to C₁₂ substituted alkyl and n is 0 to 4; and
R₅ is C₁ to C₁₂ alkyl, C₁ to C₁₂ substituted alkyl, phenyl, substituted phenyl, naphthyl, substituted naphthyl, heterocycle, substituted heterocycle, cyclic C₂ to C₇ heteroalkylene or substituted cyclic C₂ to C₇ heteroalkylene.

In a preferred embodiment, R is C₁ to C₆ alkyl or C₇ to C₁₈ phenylalkyl. In another preferred embodiment, R₂, R₃, R₄ and R₆ are, independently, hydrogen, C₁ to C₆ alkyl or halo.

In yet another embodiment, R₇ and R₈ are, independently, C₁ to C₆ alkyl, C₁ to C₆ substituted alkyl or C₇ to C₁₈ phenylalkyl. Alternatively, in an additional preferred embodiment, NR₇R₈ form a heterocyclic ring of 4 to 8 atoms, containing 0 to 3 additional heteroatoms selected from N, O or S, as well as the groups C═O, or C═NR₉, and the heterocyclic ring may be substituted, or have an additional fused ring. Preferred examples of heterocyclic NR₇R₈ include but are not limited to morpholin-4-yl, piperazin-1-yl, 3-oxopiperzin-1-y, 1 homopiperazin-1-yl, imidazole-1-yl, 2-oxoimadazolidin-1-yl, 2-oxoimidazolin-1-yl, and 2-iminioimadazolidin-1-yl.

In another preferred embodiment, R₅ is C₁ to C₁₂ alkyl, C₁ to C₁₂ substituted alkyl, phenyl, substituted phenyl, naphthyl, substituted naphthyl, heteroaryl or substituted heteroaryl. In an additional preferred embodiment, R₉ and R₁₀ are, independently, hydrogen or C₁ to C₆ alkyl.

It is to be understood that these novel compounds described above can be prepared in novel salt forms, as described herein.

Compounds of the present invention (see “Compound 1” below) can be prepared following the examples in U.S. Pat. Nos. 2,515,912 and 2,925,417. Alternatively compounds of the invention can be prepared by the alkylating Compound 2 to result Compound 1.

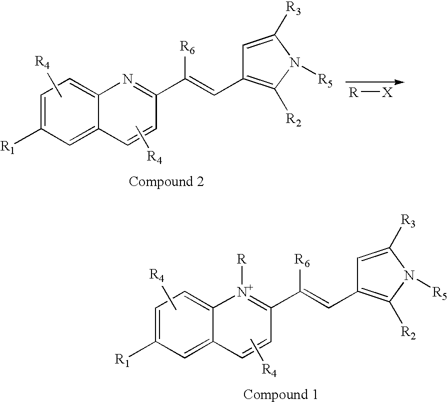

Compound 2 can be prepared by known methods for the preparation of di and tri substituted olefins.

For example, 2-vinyl quinolines have been prepared by the Pd mediated coupling of vinylstananes.²⁶ Other examples of the production of 2-vinylquinolines by the reaction of aldehydes and quinaldines in acetic anhydride, the reaction of the quinolin-2-ylmethylphosphorane and an aldehyde, or the reaction of quinolin-2-ylmethylphosphonate esters with aldehydes are found in the thesis of AG Montalban.²⁷

The present invention further provides novel quinolinium compound derivatives with the following formula:

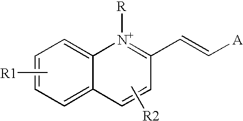

### Wherein:

A is a phenyl, substituted phenyl, heteroaryl or substituted heteroaryl, where the substitutions on the phenyl or heteroaryl can also include —OR₃, —CHO, —CN, —SO2NR3R4, —NR₅CONR₃R₄, —OCONR₃R₄, —CONR₃R₄CO₂R₃, —R5, —OR5, —SR5 R is C₁ to C₁₂ alkyl, C₁ to C₁₂ substituted alkyl, phenyl, substituted phenyl, C₇ to C₁₈ phenylalkyl or C₇ to C₁₈ substituted phenylalkyl;
R₁ is a substitution at one or more of the open positions of the benzo ring and, if at more than one position, can be the same or a different substitution;
R₂ is a substitution at one or more of the open positions of the pyridine ring and, if at more than one position, can be the same or a different substitution;
R₁ and R₂ are, independently, hydrogen, C₁ to C₁₂ alkyl, C₁ to C₁₂ substituted alkyl, heterocycle, substituted heterocycle, cyclic C₂ to C₇ heteroalkylene or substituted cyclic C₂ to C₇ heteroalkylene, bicyclic heterocycle, substituted bicyclic heterocycle, halo, —CHO or cyano, or one of the following formulas: —OR₃, —NR₃R₄, —SO2NR₃R₄, —NR₅CONR₃R₄, —OCONR₃R₄, —C(O)NR₃R₄ and —CO₂R₃; and
R₃ and R₄ are, independently, hydrogen, C₁ to C₁₂ alkyl, C₂ to C₁₂ alkenyl, C₂ to C₁₂ alkynyl, C₁ to C₁₂ substituted alkyl, C₂ to C₁₂ substituted alkenyl, C₂ to C₁₂ substituted alkynyl, C₇ to C₁₈ phenylalkyl or C₇ to C₁₈ substituted phenylalkyl: and
R5 is phenyl, substituted phenyl, heteroaryl or substituted heteroaryl, alkylphenyl, substituted alkylphenyl, alkylheteroaryl or substituted alkylheteroaryl.

Preferably, when A is substituted, it is with a phenyl, substituted phenyl, heteroaryl or substituted heteroaryl. More preferably, such phenyl or heteroaryl are substituted with C₁ to C₁₂ alkyl, C₁ to C₁₂ substituted alkyl OR₃, OR₅, SR₅, halo, —CHO, —CN, —CONR₃R₄ or —CO₂R₃. Moreover, such substitutions can be with 1 to 4 additional groups, preferably, C₁ to C₁₂ alkyl, C₁ to C₁₂ substituted alkyl OR₃, OR₅, SR₅, halo, —CHO, —CN, —CONR₃R₄ or —CO₂R₃.

In an embodiment of the invention, the above-described compounds are encompassed, excluding those compounds where R is C₁ to C₄ alkyl or hydroxyethyl. More preferably, C₁ to C₆ alkyl and hydroxyalkyl are also excluded.

In another embodiment, the above-described compounds are encompassed, excluding those compounds where R₁ methyl, dimethylamino, methoxy or heptylamino. In another embodiment, ethyl is excluded and, more preferably is C₁ to C₄ alkyl is excluded. In another embodiment, diethylamino is excluded and, more preferably, dialkylamino. In a further embodiment, ethoxy is excluded and, more preferably, alkoxy. In yet another embodiment, alkylamino is excluded. Preferably, any one or more of these exclusions is at the 7-position of the depicted quinolyl ring.

In a further embodiment, A is not a trisubstituted pyrrole, where the substitution at the 2- and 5-positions of the pyrrole is methyl or, more preferably, C₁ to C₄ alkyl, and where the 1-position of the pyrrole is phenyl, alkyl cyclohexyl or methoxyethyl. More preferably, the 1-position is not alkoxyethyl.

In the methods described below, all compounds are within the scope of the subject invention, as well as the subgenera described above. Preferably, A is pyrrole or substituted pyrrole. Preferred pyrrole substitutions include C₁ to C₁₂ alkyl, preferably methyl, preferably at the 2- or 5-positions of the pyrrole; C₁ to C₁₂ alkyl, preferably butyl, or phenyl or heteroaryl, preferably, pyridyl, more preferably, 3-pyridyl, all preferably at the 1-position of the pyrrole, and preferably —CHO, —CO₂R₃, —CONR₃R₄ or —CN, all preferably at the 4-position of the pyrrole.

Preferably, R₁ is a single specific substitution on one of the open positions of the benzo ring. More preferably, R₁ is a single specific substitution at the 6- or 7-position of the quinolyl and, even more preferably, at the 6-position (see the examples below). Preferably, R₂ is a single specific substitution on one of the open positions of the pyridine ring.

Also preferably, R₁ is a dialkylamino, preferably dimethylamino, or a halo, preferably bromo or chloro, or an alkoxy, preferably methoxy, or alkyl, preferably methyl, all preferably at the 6- or 7-position of the quinolyl; R₂ is preferably hydrogen; and R is preferably alkyl, more preferably methyl or ethyl.

Moreover, in both the compositions and methods of the claimed invention, preferably, A is an optionally substituted phenyl, thiophenyl, furanyl, indolyl, imidazolyl and isoxazolyl. Preferred substitutions include C₁ to C₁₂ alkyl, preferably methyl or butyl, or phenyl or benzyl, or -phenoxy, or dialkylamino, preferably dimethylamino, or alkoxy, preferably butyloxy. Preferably, such compounds have an amino or substituted amino at R₁, more preferably, an alkyl- or dialkylamino and, even more preferably, a dimethylamino. Also preferably, such compounds have an alkyl at R and, more preferably, methyl.

Also preferably, R₁ is a dialkylamino, preferably dimethylamino, or a halo, preferably bromo or chloro, or an alkoxy, preferably methoxy, or alkyl, preferably methyl, all preferably at the 6- or 7-position of the quinolyl; R₂ is preferably hydrogen; and R is preferably alkyl, more preferably methyl or ethyl.

The compounds of the subject invention can be used to reduce cancer cell growth. For instance, the compound shown in Example 1 reduced cancer cell growth in various cells in liquid culture without added glucose, as well as in soft agar culture. See Example 60 and Table 4; and Example 61 and Table 5. Moreover, the compounds shown in Examples 25 to 60 (except compound 53) also reduced cancer cell growth in various cell lines. See Example 60 and Table 3.

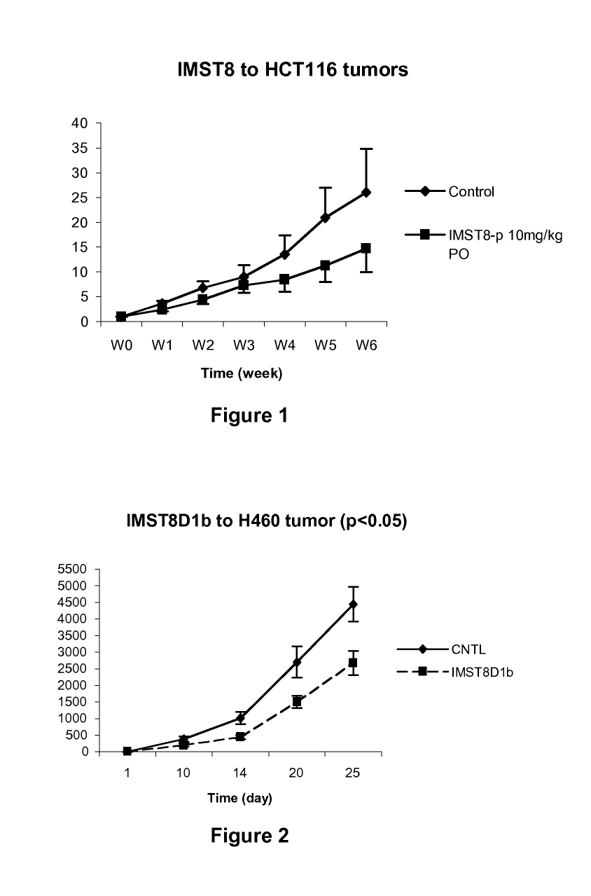

In addition, the compounds of the subject invention can be used to reduce tumor size in vivo. For example, the compound of Example 1 reduced the size of colon cancer tumors in a mice xenograft advanced stage model. See Example 62 and FIG. 1. Furthermore, the compound of Example 56 reduced the size of non-small lung cancer tumors in a mice xenograft early stage model. See Example 63 and FIG. 2. Moreover, the compounds of Examples 36 and 47 reduced or suppressed lung cancer. See Example 66 and FIG. 6.

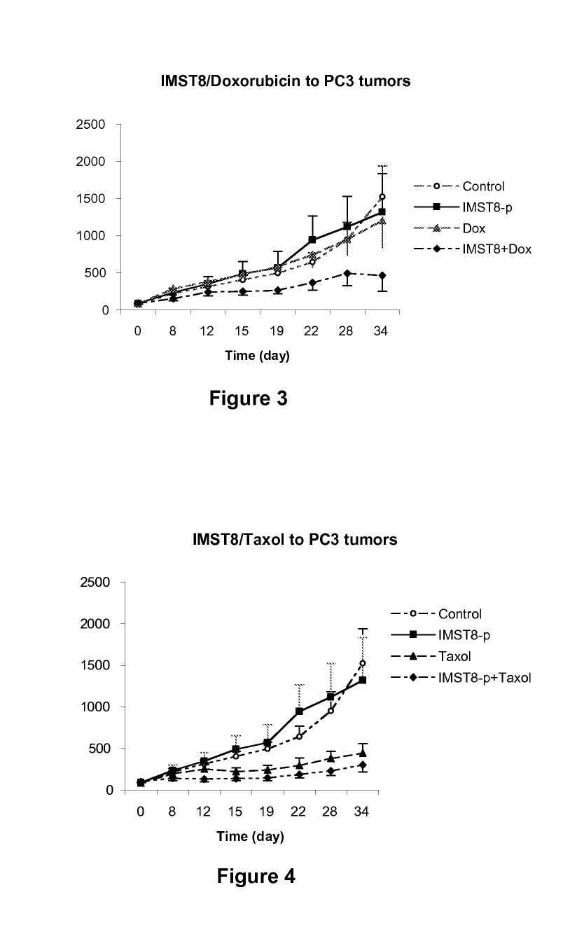

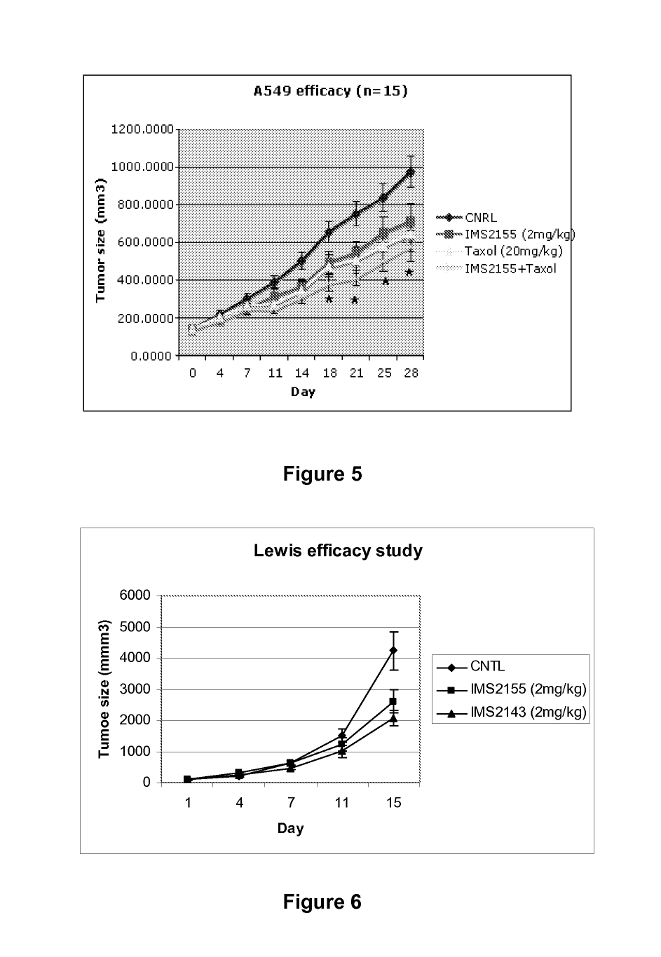

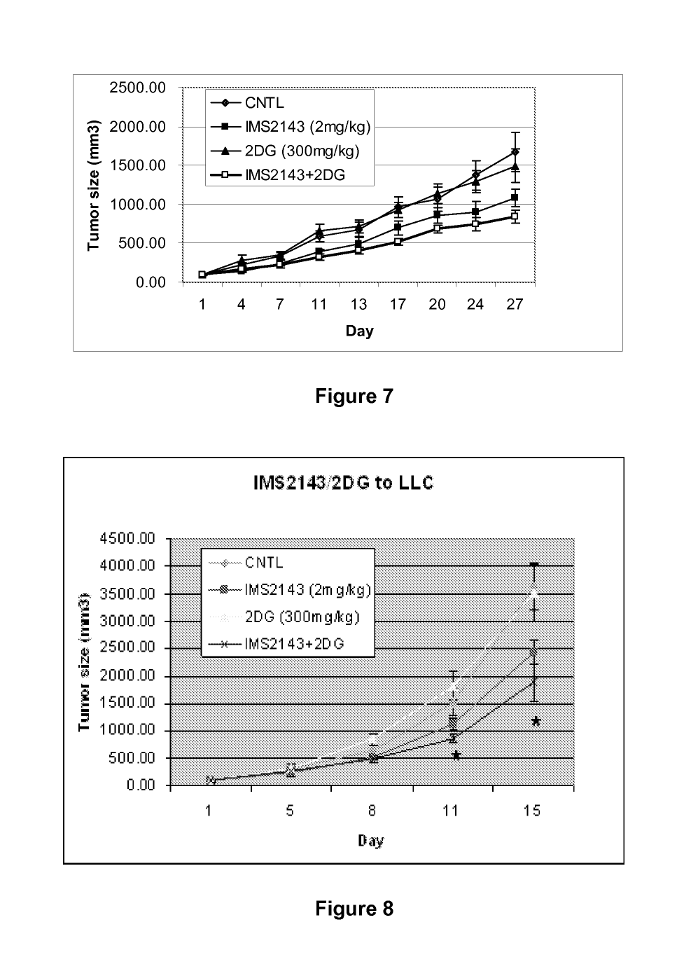

The present invention also provides a combination of a) a compound of the invention with b) one or more additional active chemotherapeutic agents. A preferred additional active chemotherapeutic agent includes but is not limited to taxol, doxorubicin or 2DG. As shown in FIG. 3 and described in Example 64A and Table 6, the combination of the compound of Example 1 and doxorubicin was very effective in inhibiting prostate tumor growth in a mice xenograft model. Moreover, as shown in FIG. 4 and described in Example 64B and Table 6, the combination of the compound of Example 1 and taxol was also very effective in inhibiting prostate tumor growth in a mice xenograft model. Similarly, the combination of the compound of Example 47 and taxol was synergistically effective in reducing lung tumors. See Example 65 and FIG. 5. In addition, the combination of the compound of Example 36 and 2DG had a synergistic anti-tumor effect in two lung cancer models. See Example 67 and FIGS. 7 and 8.

Other prophylactic or therapeutic agents that are anti-cancer agents that can be used with a compound of the invention include, but are not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, J dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferonbeta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin and zorubicin hydrochloride.

Further anti-cancer drugs that can be used with a compound of the invention include, but are not limited to: 20-epi-1, 25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthioninesulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabineocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflornithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetiumtexaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor1-based therapy, mustard anticancer agent, mycaperoxide B, mycobacterial cell wall extract, myriaporone, N-acetyldinaline, N-substitutedbenzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, 06-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras farnesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, syntheticglycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, zinostatin stimalamer, 5-fluorouracil, leucovorin, angiostatin (plasminogen fragment), antiangiogenic antithrombin III, Angiozyme, ABT-627, Bay 12-9566, Benefin, Bevacizumab, BMS-275291, cartilage-derived inhibitor(CDI), CAI, CD59 complement fragment, CEP-7055, Col 3, Combretastatin A-4, Endostatin (collagen XVIII fragment), fibronectin fragment, Gro-beta, Halofuginone, Heparinases, Heparin hexasaccharide fragment, HMV833, Human chorionicgonadotropin (hCG), IM-862, Interferonalpha/beta/gamma, Interferon inducible protein (IP-10), Interleukin-12, Kringle 5 (plasminogen fragment), Marimastat, Metalloproteinase inhibitors (TIMPs), 2-Methoxyestradiol, MMI 270 (CGS 27023A), MoAbIMC-1C11, Neovastat, NM-3, Panzem, PI-88, Placental ribonuclease inhibitor, Plasminogen activator inhibitor, Platelet factor-4 (PF4), Prinomastat, Prolactin16 kD fragment, Proliferin-related protein (PRP), PTK 787/ZK 222594, Retinoids, Solimastat, Squalamine, SS 3304, SU 5416, SU6668, SU11248, Tetrahydrocortisol-S, tetrathiomolybdate, thalidomide, Thrombospondin-1 (TSP-1), TNP-470, Transforming growth factor-beta (TGF-b), Vasculostatin, Vasostatin (calreticulin fragment), ZD6126, ZD 6474, farnesyltransferase inhibitors(FTI) and bisphosphonates.

A preferred class of anti-cancer agent that can be used with a compound of the invention is a kinase inhibitor. Kinase inhibitors that can be used include, but are not limited to, inhibitors of ABL, ACK, AFK, AKT (e.g., AKT-1, AKT-2, and AKT-3), ALK, AMP-PK, ATM, Aurora1, Aurora2, bARKl1bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAKI, IRAK2, IRK, ITK, LCK, LOK, LYN, MAPK, MAPKAPK-1, MAPKAPK-2, MEK, MET, MFPK, MHCK, MLCK, MLK3, NEU, NIK, PDGF receptor alpha, PDGF receptor beta, PHK, PI-3 kinase, PKA, PKB, PKC, PKG, PRK1, PYK2, p38 kinases, p135tyk2, p34cdc2, p42cdc2, p42mapk, p44mpk, RAF, RET, RIP, RIP-2, RK, RON, RS kinase, SRC, SYK, S6K, TAK1, TEC, TIE1, TIE2, TRKA, TXK, TYK2, UL13, VEGFR1, VEGFR2, YES, YRK, ZAP-70, and all subtypes of these kinases (see e.g., Hardie and Hanks (1995) The Protein Kinase Facts Book, I and II, Academic Press, San Diego, Calif.).

A compound of the invention can also be used with one or more agents that are angiogenesis inhibitors such as, but not limited to: Angiostatin (plasminogen fragment), antiangiogenic antithrombin III, Angiozyme, ABT-627, Bay 12-9566, Benefin, Bevacizumab, BMS-275291, cartilage-derived inhibitor (CDI), CAI, CD59 complement fragment, CEP-7055, Col 3, Combretastatin A-4, Endostatin (collagen XVIII fragment), fibronectin fragment, Gro-beta, Halofuginone, Heparinases, Heparin hexasaccharide fragment, HMV833, Human chorionic gonadotropin (hCG), IM-862, Interferon alpha/beta/gamma, Interferon inducible protein (IP-10), hterleukin-12, Kringle 5 (plasminogen fragment), Marimastat, Metalloproteinase inhibitors (TIMPs), 2-Methoxyestradiol, MMI 270 (CGS 27023A), MoAbIMC-1C11, Neovastat, NM-3, Panzem, PI-88, Placental ribonuclease inhibitor, Plasminogen activator inhibitor, Platelet factor-4 (PF4), Prinomastat, Prolactin 16 kD fragment, Proliferin-related protein (PRP), PTK 787/ZK 222594, Retinoids, Solimastat, Squalamine, SS 3304, SU 5416, SU6668, SU11248, Tetrahydrocortisol-S, tetrathiomolybdate, thalidomide, Thrombospondin-1 (TSP-1), TNP-470, Transforming growth factor-beta(TGF-), Vasculostatin, Vasostatin (calreticulin fragment), ZD6126, ZD6474, farnesyl transferase inhibitors(FTI) and bisphosphonates.

The present invention also contemplates pharmaceutical compositions that include a pharmaceutically acceptable carrier and, as an active ingredient, the novel compounds or salt forms of the invention, as described above. Preferably, in the pharmaceutical composition the active ingredient is delivered in any acceptable tablet or capsule form, or is dissolved an aquous or lipid carrier. Further, in accordance with a preferred embodiment of the present invention, the composition additionally comprises at least one other chemotherapeutic agent.

The present invention additionally provides a method for reduction of the growth of mammalian cancer cells, comprising applying to the cancer cells a therapeutically effective amount of the novel compound or salt form of the invention, as described herein.

Furthermore, the present invention provides a method for the treatment of mammalian cancer, comprising administering to the mammalian subject a pharmaceutical composition containing as an active ingredient a therapeutically effective amount of the novel compound or salt form of the invention, as described herein.

The term “reduction of growth” in relation to cancer cells, in the context of the present invention refers to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a more differentiated cell type to a less differentiated cell type; a deceleration in the neoplastic progress; or alternatively the slowing of the progression of the cancer cells from one stage to the next.

Reduction of growth of cancer cells may be utilized for the treatment of cancer by the administration, to an individual in need of such treatment, of a therapeutically effective amount of the compound of the present invention, as described herein.

The present invention additionally discloses use of a composition of the invention, as described above, for preparing a medicament for the treatment of cancer in mammals.

The term “treatment of cancer” in the context of the present invention includes at least one of the following: a decrease in the rate of growth of the cancer (i.e. the cancer still grows but at a slower rate); cessation of growth of the cancerous growth, i.e., stasis of the tumor growth, and, in preferred cases, the tumor diminishes or is reduced in size. The term also includes reduction in the number of metastasis, reduction in the number of new metastasis formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment. This term also encompasses prevention for prophylactic situations or for those individuals who are susceptible to contracting a tumor. The administration of the compounds of the present invention will reduce the likelihood of the individual contracting the disease. In preferred situations, the individual to whom the compound is administered does not contract the disease.

The term “cancer” in the context of the present invention includes all types of neoplasm whether in the form of solid or non-solid tumors, from all origins, and includes both malignant and benign conditions as well as their metastasis. In particular this term refers to: carcinoma, sarcoma, adenoma, hepatocellular carcinoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, lymphagiosarcoma, synovioama, Ewing's tumor, leimyosarcoma, rhabdotheliosarcoma, colon carcinoma, pancreatic cancer, breast cancer, ovarian cancer, prostate cancer, squamous cell carcinoma, basal cell carcinoma, adenocarcinoma, renal cell carcinoma, hematoma, bile duct carcinoma, melanoma, choriocarcinoma, seminoma, embryonal carcinoma, Wilms' tumor, cervical cancer, testicular tumor, lung carcinoma, small cell and non-small cell lung carcinoma, bladder carcinoma, epithelial carcinoma, glioma, astrocyoma, medulloblastoma, craniopharyngioma, ependynoma, pinealoma, retinoblastoma, multiple myeloma, rectal carcinoma, cancer of the thyroid, head and neck cancer, brain cancer, cancer of the peripherial nervous system, cancer of the central nervous system, neuroblastoma, cancer of the edometrium, myeloid lymphoma, leukemia, lymphoma, lymphoproliferative diseases, acute myelocytic leukemia, chronic leukemia, Hodgkin's lymphoma, non-Hodgkin's lymphoma as well as metastasis of all the above.

More preferably, the cancer is selected from the group consisting of prostate cancer, breast cancer, skin cancer, colon cancer, lung cancer and pancreatic cancer.

In other embodiments of the use of preparing a medicament, the medicament additionally comprises at least one active chemotherapeutic agent other than the compound of the invention. In certain embodiments, the novel compound may be administered alongside with traditional chemotherapeutic drugs that are effective but have considerable side effects. The combination of a compound of the invention and the traditional drug may allow administration of a lesser quantity of the traditional drug, and thus the side effects experienced by the subject may be significantly lower, while a sufficient chemotherapeutic effect is nevertheless achieved. Preferred additional active chemotherapeutics include but are not limited to taxol or doxorubicin.

The present invention additionally discloses a method for the treatment of cancer in mammals, comprising administering to a mammal a therapeutically effective amount of a pharmaceutical composition comprising as the active ingredient a compound of the present invention, as described above. Additionally, in a preferred embodiment of the method, the compound is administered at a dosage selected from 1 μg-1000 mg/kg body weight.

There is also provided in the present invention a pharmaceutical composition for the treatment of cancer in mammals, comprising as the active ingredient a therapeutically effective amount of a compound of the invention, as described above.

There is also provided in the present invention a pharmaceutical composition for the treatment of cancer in mammals, comprising as the active ingredient a therapeutically effective amount of a compound of the invention, as described herein, and a pharmaceutically acceptable carrier.

The term “pharmaceutically acceptable” means approved by a regulatory agency of the Federal or a state government or listed in the U.S. Pharmacopoeia or other generally recognized pharmacopoeia for use in animals and, more particularly, in humans.

The term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, cyclodextrins and the like.

The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

The compositions can take the form of solutions, suspensions, emulsion, tablets, pills, capsules, powders, sustained-release formulations and the like. The composition can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of a compound of the invention, preferably in a substantially purified form, together with a suitable amount of carrier so as to provide the form for proper administration to the subject.

The carrier can be selected at times based on the desired form of the formulation. The carrier may also at times have the effect of the improving the delivery or penetration of the active ingredient to the target tissue, for improving the stability of the drug, for slowing clearance rates, for imparting slow release properties, for reducing undesired side effects etc. The carrier may also be a substance that stabilizes the formulation (e.g. a preservative), for providing the formulation with an edible flavor, etc.

The carriers may be any of those conventionally used and are limited only by chemical-physical considerations, such as solubility and lack of reactivity with the compound of the invention, and by the route of administration. The choice of carrier will be determined by the particular method used to administer the pharmaceutical composition. Accordingly, the carrier may include additives, colorants, diluents, buffering agents, disintegrating agents, moistening agents, preservatives, flavoring agents, and pharmacologically compatible carriers. In addition, the carrier may be an adjuvant, which, by definition are substances affecting the action of the active ingredient in a predictable way.

Methods of introduction of a pharmaceutical composition comprising a compound of the invention include, but are not limited to, topical, intradermal, intramuscular, intraperitoneal, intravenous, subcutaneous, intranasal, epidural, ophthalmic, and oral routes. The compounds may be administered by any convenient route, for example by infusion or bolus injection, by absorption through epithelial linings (e.g., oral mucosa, rectal and intestinal mucosa, etc.), and may be administered together with other therapeutically active agents. It is preferred that administration is localized, but it may be systemic. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer, and formulation with an aerosolizing agent.

It may be desirable to administer the pharmaceutical composition of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material. According to some preferred embodiments, administration can be by direct injection e.g., via a syringe, at the site of a tumor or neoplastic or pre-neoplastic tissue.

Pharmaceutical compositions suitable for oral administration may consist of (a) liquid solutions, where an effective amount of the active substance is dissolved in diluents, such as water, saline, natural juices, alcohols, syrups, etc.; (b) solid dosage forms such as capsules (e.g. the ordinary hard- or soft-shelled gelatin type containing, for example, surfactants, lubricants, and inert fillers), tablets, lozenges (wherein the active substance is flavored, such as with sucrose and acacia or tragacanth, or the active substance is in an inert base, such as gelatin and glycerin), and troches, each containing a predetermined amount of the active ingredient as solids or granules; (c) powders; (d) suspensions in an appropriate liquid; (e) suitable emulsions; (f) liposome formulation; and others.

In yet another embodiment, the composition is prepared for topical administration, e.g. as an ointment, a gel a drop or a cream. For topical administration to body surfaces using, for example, creams, gels, drops, ointments and the like, the compounds of the present invention can be prepared and applied in a physiologically acceptable diluent with or without a pharmaceutical carrier. The present invention may be used topically or transdermally to treat cancer, for example, melanoma. Adjuvants for topical or gel base forms may include, for example, sodium carboxymethylcellulose, polyacrylates, polyoxyethylene-polyoxypropylene-block polymers, polyethylene glycol and wood wax alcohols.

For directed internal topical applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; or a glidant such as colloidal silicon dioxide. When the dosage unit form is a capsule, it can contain, in addition to materials of the above type, a liquid carrier such as a fatty oil. In addition, dosage unit forms can contain various other materials which modify the physical form of the dosage unit, for example, coatings of sugar, shellac, or other enteric agents.

A compound of the present invention can be delivered in a controlled release system. In one embodiment, an infusion pump may be used to administer a compound of the invention, such as one that is used for delivering insulin or chemotherapy to specific organs or tumors (see Buchwald et al., 1980, Surgery 88: 507; Saudek et al., 1989, N. Engl. J. Med. 321: 574). In a preferred form, a compound of the invention is administered in combination with a biodegradable, biocompatible polymeric implant, which releases the compound over a controlled period of time at a selected site. Examples of preferred polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). In yet another embodiment, a controlled release system can be placed in proximity of the therapeutic target, thus requiring only a fraction of the systemic dose.

At times, the active compound may be made into aerosol formulations to be administered via inhalation. These aerosol formulations can be placed into pressurized acceptable propellants, such as propane, butane, nitrogen, and the like. They also may be formulated as pharmaceuticals for non-pressured preparations, such as in a nebulizer or an atomizer.

Furthermore, at times, the pharmaceutical compositions may be formulated for parenteral administration (subcutaneous, intravenous, intra-arterial, or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. The above formulations may also be used for direct intra-tumoral injection. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described and known in the art.

The amount of a compound of the invention that will be effective in the treatment of a particular disorder or condition, including cancer, will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. A preferred dosage will be within the range of 0.01-1000 mg/kg of body weight, more preferably, 0.1 mg/kg to 100 mg/kg and even more preferably 1 mg/kg to 10 mg/kg. Effective doses may be extrapolated from dose-response curves derived from in vitro or animal model test bioassays or systems.

A “therapeutic” treatment is a treatment administered to a subject who exhibits signs of pathology for the purpose of diminishing or eliminating those signs. A “therapeutically effective amount” of a compound of the invention is that amount of compound which is sufficient to provide a beneficial effect to the subject to which the compound is administered.

Patients in need thereof may suffer from a disease such as cancer or may have been determined to have a greater susceptibility to such disease. Thus, the method of treatment according to the present invention includes both therapeutic and prophylactic utility.

A compound of the invention can be tested in vivo for the desired therapeutic or prophylactic activity as well as for determination of a therapeutically effective dosage. For example, such compounds can be tested in suitable animal model systems prior to testing in humans, including, but not limited to, rats, mice, chicken, cows, monkeys, rabbits, and the like. For in vivo testing, prior to administration to humans, any animal model system known in the art may be used.

When the above-described compounds include one or more chiral centers, the stereochemistry of such chiral centers can independently be in the R or S configuration, or a mixture of the two. The chiral centers can be further designated as R or S or R,S or d,D, l,L or d,l, D,L.

Regarding the compounds and described herein, the suffix “ene” added to any of the described terms means that two parts of the substituent are each connected to two other parts in the compound (unless the substituent contains only one carbon, in which case such carbon is connected to two other parts in the compound, for example, methylene).

The term “C₁ to C₁₂ alkyl” denotes such radicals as methyl, ethyl, n-propyl, isopropyl, n-butyl, iso-butyl, sec-butyl, tert-butyl, amyl, tert-amyl, hexyl, heptyl, octyl, nonyl, decyl, undecyl, dodecyl and the like. Preferred “C₁ to C₁₂ alkyl” groups are methyl, ethyl, iso-butyl, sec-butyl and iso-propyl. Similarly, the term “C₁ to C₁₂ alkylene” denotes radicals of 1 to 12 carbons connected to two other parts in the compound.

The term “C₂ to C₁₂ alkenyl” denotes such radicals as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl, (as well as octenyl, nonenyl, decenyl, undecenyl, dodecenyl radicals attached at any appropriate carbon position and the like) as well as dienes and trienes of straight and branched chains.

The term “C₂ to C₁₂ alkynyl” denotes such radicals as ethanol, propynyl, 2-butynyl, 2-pentynyl, 3-pentynyl, 2-hexynyl, 3-hexynyl, 4-hexynyl, 2-heptynyl, 3-heptynyl, 4-heptynyl, 5-heptynyl (as well as octynyl, nonynyl, decynyl, undecynyl, dodecynyl radicals attached at any appropriate carbon position and the like) as well as di- and tri-ynes of straight and branched chains.

The terms “C₁ to C₁₂ substituted alkyl,” “C₂ to C₁₂ substituted alkenyl,” “C₂ to C₁₂ substituted alkynyl,” “C₁ to C₁₂ substituted alkylene,” “C₂ to C₁₂ substituted alkenylene” and “C₂ to C₁₂ substituted alkynylene” denote groups that are substituted by one or more, and preferably one or two, halogen, hydroxy, protected hydroxy, oxo, protected oxo, C₃ to C₇ cycloalkyl, phenyl, naphthyl, amino, protected amino, (monosubstituted)amino, protected (monosubstituted)amino, (disubstituted)amino, guanidino, protected guanidino, heterocyclic ring, substituted heterocyclic ring, imidazolyl, indolyl, pyrrolidinyl, C₁ to C₁₂ alkoxy, C₁ to C₁₂ acyl, C₁ to C₁₂ acyloxy, nitro, carboxy, protected carboxy, carbamoyl, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, cyano, methylsulfonylamino, thiol, C₁ to C₁₀ alkylthio or C₁ to C₁₀ alkylsulfonyl groups. The substituted alkyl groups may be substituted once or more, and preferably once or twice, with the same or with different substituents. A preferred substitution is halo.

The term “protected oxo” denotes a carbon atom bonded to two additional carbon atoms substituted with two alkoxy groups or twice bonded to a substituted diol moiety, thereby forming an acyclic or cyclic ketal moiety.

The term “C₁ to C₁₂ alkoxy” as used herein denotes groups such as methoxy, ethoxy, n-propoxy, isopropoxy, n-butoxy, t-butoxy and like groups. A preferred alkoxy is methoxy. The term “C₁ to C₁₂ substituted alkoxy” means the alkyl portion of the alkoxy can be substituted in the same manner as in relation to C₁ to C₁₂ substituted alkyl. A preferred substitution is halo. Similarly, the term “C₁ to C₁₂ phenylalkoxy” as used herein means “C₁ to C₁₂ alkoxy” bonded to a phenyl radical.

The term “C₁ to C₁₂ acyloxy” denotes herein groups such as formyloxy, acetoxy, propionyloxy, butyryloxy, pivaloyloxy, pentanoyloxy, hexanoyloxy, heptanoyloxy, octanoyloxy, nonanoyloxy, decanoyloxy, undecanoyloxy, dodecanoyloxy and the like.

Similarly, the term “C₁ to C₁₂ acyl” encompasses groups such as formyl, acetyl, propionyl, butyryl, pentanoyl, pivaloyl, hexanoyl, heptanoyl, octanoyl, nonanoyl, decanoyl, undecanoyl, dodecanoyl, benzoyl and the like. Preferred acyl groups are acetyl and benzoyl.

The term “C₁ to C₁₂ substituted acyl” denotes the acyl group substituted by one or more, and preferably one or two, halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C₁ to C₁₂ alkoxy, C₁ to C₁₂ acyl, C₁ to C₁₂ acyloxy, nitro, C₁ to C₁₂ alkyl ester, carboxy, protected carboxy, carbamoyl, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, cyano, methylsulfonylamino, thiol, C₁ to C₁₀ alkylthio or C₁ to C₁₀ alkylsulfonyl groups. The substituted acyl groups may be substituted once or more, and preferably once or twice, with the same or with different substituents.

The substituent term “C₃ to C₇ cycloalkyl” includes the cyclopropyl, cyclobutyl, cyclopentyl, cyclohexyl or cycloheptyl rings. Similarly, a substituent that can be C₃ to C₇ cycloalkyl” can also be “C₅ to C₇ cycloalkyl,” which includes the cyclopentyl, cyclohexyl or cycloheptyl rings.

The substituent term “C₃ to C₇ substituted cycloalkyl” or “C₅ to C₇ substituted cycloalkyl” indicates the above cycloalkyl rings substituted by one or two halogen, hydroxy, protected hydroxy, C₁ to C₁₀ alkylthio, C₁ to C₁₀ alkylsulfoxide, C₁ to C₁₀ alkylsulfonyl, C₁ to C₁₀ substituted alkylthio, C₁ to C₁₀ substituted alkylsulfoxide, C₁ to C₁₀ substituted alkylsulfonyl, C₁ to C₁₂ alkyl, C₁ to C₁₂ alkoxy, C₁ to C₁₂ substituted alkyl, C₁ to C₁₂ alkoxy, oxo, protected oxo, (monosubstituted)amino, (disubstituted)amino, trifluoromethyl, carboxy, protected carboxy, phenyl, substituted phenyl, phenylthio, phenylsulfoxide, phenylsulfonyl, amino, or protected amino groups.

The term “cycloalkylene” means a cycloalkyl, as defined above, where the cycloalkyl radical is bonded at two positions connecting together two separate additional groups. Similarly, the term “substituted cycloalkylene” means a cycloalkylene where the cycloalkyl radical is bonded at two positions connecting together two separate additional groups and further bearing at least one additional substituent.

The term “C₅ to C₇ cycloalkenyl” indicates a 1,2, or 3-cyclopentenyl ring, a 1, 2, 3 or 4-cyclohexenyl ring or a 1, 2, 3, 4 or 5-cycloheptenyl ring, while the term “substituted C₅ to C₇ cycloalkenyl” denotes the above C₅ to C₇ cycloalkenyl rings substituted by a C₁ to C₁₂ alkyl radical, halogen, hydroxy, protected hydroxy, C₁ to C₁₂ alkoxy, trifluoromethyl, carboxy, protected carboxy, oxo, protected oxo, (monosubstituted)amino, protected (monosubstituted)amino, (disubstituted)amino, phenyl, substituted phenyl, amino, or protected amino.

The term “C₅ to C₇ cycloalkenylene” is a cycloalkenyl ring, as defined above, where the cycloalkenyl radical is bonded at two positions connecting together two separate additional groups. Examples of C₅ to C₇ cycloalkenylenes include 1,3-cyclopentylene and 1,2-cyclohexylene.

Similarly, the term “substituted C₅ to C₇ cycloalkenylene” means a cycloalkenylene further substituted by halogen, hydroxy, protected hydroxy, C₁ to C₁₀ alkylthio, C₁ to C₁₀ alkylsulfoxide, C₁ to C₁₀ alkylsulfonyl, C₁ to C₁₀ substituted alkylthio, C₁ to C₁₀ substituted alkylsulfoxide, C₁ to C₁₀ substituted alkylsulfonyl, C₁ to C₁₂ alkyl, C₁ to C₁₂ alkoxy, C₁ to C₁₂ substituted alkyl, C₁ to C₁₂ alkoxy, oxo, protected oxo, (monosubstituted)amino, (disubstituted)amino, trifluoromethyl, carboxy, protected carboxy, phenyl, substituted phenyl, phenylthio, phenylsulfoxide, phenylsulfonyl, amino, or protected amino group.

The term “heterocycle” or “heterocyclic ring” denotes optionally substituted five-membered to eight-membered rings that have 1 to 4 heteroatoms, such as oxygen, sulfur and/or nitrogen, in particular nitrogen, either alone or in conjunction with sulfur or oxygen ring atoms. The term “bicyclic heterocycle” means two such rings fused to each other. These five-membered to eight-membered rings may be saturated, fully unsaturated or partially unsaturated, with fully saturated rings being preferred. Preferred heterocyclic rings include morpholino, piperidinyl, piperazinyl, 2-amino-imidazoyl, tetrahydrofurano, pyrrolo, tetrahydrothiophen-yl, hexylmethyleneimino and heptylmethyleneimino.

The terms “substituted heterocycle” or “substituted heterocyclic ring” and “substituted bicyclic heterocycle” mean the above-described heterocyclic or fused biheterocyclic rings are substituted with, for example, one or more, and preferably one or two, substituents which are the same or different which substituents can be halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N-((C₁ to C₁₂ alkyl)sulfonyl)amino, N-(phenylsulfonyl)amino, heterocycle or substituted heterocycle groups.

The term “heteroaryl” means a heterocyclic aromatic derivative which is a five-membered or six-membered ring system having from 1 to 4 heteroatoms, such as oxygen, sulfur and/or nitrogen, in particular nitrogen, either alone or in conjunction with sulfur or oxygen ring atoms. Examples of heteroaryls include pyridinyl, pyrimidinyl, and pyrazinyl, pyridazinyl, pyrrolo, furano, oxazolo, isoxazolo, phthalimido, thiazolo and the like.

The term “substituted heteroaryl” means the above-described heteroaryl is substituted with, for example, one or more, and preferably one or two, substituents which are the same or different which substituents can be halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N-((C₁ to C₁₂ alkyl)sulfonyl)amino or N-(phenylsulfonyl)amino groups.

The term “C₇ to C₁₈ phenylalkyl” denotes a C₁ to C₁₂ alkyl group substituted at any position within the alkyl chain by a phenyl. The definition includes groups of the formula: -phenyl-alkyl, -alkyl-phenyl and -alkyl-phenyl-alkyl. Examples of such a group include benzyl, 2-phenylethyl, 3-phenyl(n-propyl), 4-phenylhexyl, 3-phenyl(n-amyl), 3-phenyl(sec-butyl) and the like. Preferred C₇ to C₁₈ phenylalkyl groups are any one of the preferred alkyl groups described herein combined with a phenyl group.

Similarly, the term “C₁ to C₁₂ heterocycloalkyl” denotes a C₁ to C₁₂ alkyl group substituted at any position within the alkyl chain by a “heterocycle,” as defined herein. The definition includes groups of the formula: -heterocyclic-alkyl, -alkyl-heterocyclic and -alkyl-heterocyclic-alkyl. Preferred C₁ to C₁₂ heterocycloalkyl groups are any one of the preferred alkyl groups described herein combined with any one of the preferred heterocycle groups described herein.

The terms “C₇ to C₁₈ substituted phenylalkyl” and “C₁ to C₁₂ substituted heterocycloalkyl” denote a C₇ to C₁₈ phenylalkyl group or C₁ to C₁₂ heterocycloalkyl substituted (on the alkyl or, where applicable, phenyl or heterocyclic portion) with one or more, and preferably one or two, groups chosen from halogen, hydroxy, protected hydroxy, oxo, protected oxo, amino, protected amino, (monosubstituted)amino, protected (monosubstituted)amino, (disubstituted)amino, guanidino, protected guanidino, heterocyclic ring, substituted heterocyclic ring, C₁ to C₁₂ alkyl, C₁ to C₁₂ substituted alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, nitro, carboxy, protected carboxy, carbamoyl, carboxamide, protected carboxamide, N—(C₁ to C₁₂ alkyl)carboxamide, protected N—(C₁ to C₁₂ alkyl)carboxamide, N,N—(C₁ to C₁₂ dialkyl)carboxamide, cyano, N—(C₁ to C₁₂ alkylsulfonyl)amino, thiol, C₁ to C₁₀ alkylthio, C₁ to C₁₀ alkylsulfonyl groups; and/or the phenyl group may be substituted with one or more, and preferably one or two, substituents chosen from halogen, hydroxy, protected hydroxy, cyano, nitro, C₁ to C₁₂ alkyl, C₁ to C₁₂ substituted alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N-((C₁ to C₁₂ alkyl)sulfonyl)amino, N-(phenylsulfonyl)amino, cyclic C₂ to C₁₂ alkylene or a phenyl group, substituted or unsubstituted, for a resulting biphenyl group. The substituted alkyl, phenyl or heterocyclic groups may be substituted with one or more, and preferably one or two, substituents which can be the same or different.

The term “C₇ to C₁₈ phenylalkylene” specifies a C₇ to C₁₈ phenylalkyl, as defined above, where the phenylalkyl radical is bonded at two different positions connecting together two separate additional groups. The definition includes groups of the formula: -phenyl-alkyl-, -alkyl-phenyl- and -alkyl-phenyl-alkyl-. Substitutions on the phenyl ring can be 1,2, 1,3 or 1,4. C₇ to C₁₈ phenylalkylenes include, for example, 1,4-tolylene and 1,3-xylylene.

Similarly, the term “C₁ to C₁₂ heterocycloalkylene” specifies a C₁ to C₁₂ heterocycloalkyl, as defined above, where the heterocycloalkyl radical is bonded at two different positions connecting together two separate additional groups. The definition includes groups of the formula: -heterocyclic-alkyl-, -alkyl-heterocyclic and -alkyl-heterocyclic-alkyl-.

The terms “C₇ to C₁₈ substituted phenylalkylene” and “C₁ to C₁₂ substituted heterocycloalkylene” means a C₇ to C₁₈ phenylalkylene or C₁ to C₁₂ heterocycloalkylene as defined above that is further substituted by halogen, hydroxy, protected hydroxy, C₁ to C₁₀ alkylthio, C₁ to C₁₀ alkylsulfoxide, C₁ to C₁₀ alkylsulfonyl, C₁ to C₁₀ substituted alkylthio, C₁ to C₁₀ substituted alkylsulfoxide, C₁ to C₁₀ substituted alkylsulfonyl, C₁ to C₁₂ alkyl, C₁ to C₁₂ alkoxy, C₁ to C₁₂ substituted alkyl, C₁ to C₁₂ alkoxy, oxo, protected oxo, (monosubstituted)amino, (disubstituted)amino, trifluoromethyl, carboxy, protected carboxy, phenyl, substituted phenyl, phenylthio, phenylsulfoxide, phenylsulfonyl, amino, or protected amino group on the phenyl ring or on the alkyl group.

The term “substituted phenyl” specifies a phenyl group substituted with one or more, and preferably one or two, moieties chosen from the groups consisting of halogen, hydroxy, protected hydroxy, cyano, nitro, C₁ to C₁₂ alkyl, C₁ to C₁₂ substituted alkyl, C₁ to C₁₂ alkoxy, C₁ to C₁₂ substituted alkoxy, C₁ to C₁₂ acyl, C₁ to C₁₂ substituted acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N-((C₁ to C₁₂ alkyl)sulfonyl)amino, N-(phenylsulfonyl)amino or phenyl, wherein the phenyl is substituted or unsubstituted, such that, for example, a biphenyl results.

The term “phenoxy” denotes a phenyl bonded to an oxygen atom, wherein the binding to the rest of the molecule is through the oxygen atom. The term “substituted phenoxy” specifies a phenoxy group substituted with one or more, and preferably one or two, moieties chosen from the groups consisting of halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N-((C₁ to C₁₂ alkyl)sulfonyl)amino and N-(phenylsulfonyl)amino.

The term “aryl” refers to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like. The term “aryloxy” refers to an “aryl” group bonded to an oxygen atom, wherein the binding to the rest of the molecule is through the oxygen atom.

The term “C₇ to C₁₈ substituted phenylalkoxy” denotes a C₇ to C₁₈ phenylalkoxy group bonded to the rest of the molecule through the oxygen atom, wherein the phenylalkyl portion is substituted with one or more, and preferably one or two, groups selected from halogen, hydroxy, protected hydroxy, oxo, protected oxo, amino, protected amino, (monosubstituted)amino, protected (monosubstituted)amino, (disubstituted)amino, guanidino, heterocyclic ring, substituted heterocyclic ring, C₁ to C₁₂ alkoxy, C₁ to C₁₂ acyl, C₁ to C₁₂ acyloxy, nitro, carboxy, protected carboxy, carbamoyl, carboxamide, protected carboxamide, N—(C₁ to C₁₂ alkyl)carboxamide, protected N—(C₁ to C₁₂ alkyl)carboxamide, N,N—(C₁ to C₁₂ dialkyl)carboxamide, cyano, N—(C₁ to C₁₂ alkylsulfonyl)amino, thiol, C₁ to C₁₀ alkylthio, C₁ to C₁₀ alkylsulfonyl groups; and/or the phenyl group can be substituted with one or more, and preferably one or two, substituents chosen from halogen, hydroxy, protected hydroxy, cyano, nitro, C₁ to C₁₂ alkyl, C₁ to C₁₂ alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N-((C₁ to C₁₂ alkyl)sulfonyl)amino, N-(phenylsulfonyl)amino or a phenyl group, substituted or unsubstituted, for a resulting biphenyl group. The substituted alkyl or phenyl groups may be substituted with one or more, and preferably one or two, substituents which can be the same or different.

The term “phthalimide” means a cyclic imide which is made from phthalic acid, also called 1,2-benzenedicarboxylic acid. The term “substituted phthalimide” specifies a phthalimide group substituted with one or more, and preferably one or two, moieties chosen from the groups consisting of halogen, hydroxy, protected hydroxy, cyano, nitro, C₁ to C₁₂ alkyl, C₁ to C₁₂ alkoxy, C₁ to C₁₂ substituted alkoxy, C₁ to C₁₂ acyl, C₁ to C₁₂ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N-((C₁ to C₁₂ alkyl)sulfonyl)amino and N-(phenylsulfonyl)amino. The term “substituted naphthyl” specifies a naphthyl group substituted with one or more, and preferably one or two, moieties either on the same ring or on different rings chosen from the groups consisting of halogen, hydroxy, protected hydroxy, cyano, nitro, C₁ to C₆ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₁₂ alkyl)carboxamide, protected N—(C₁ to C₁₂ alkyl)carboxamide, N,N-di(C₁ to C₁₂ alkyl)carboxamide, trifluoromethyl, N-((C₁ to C₁₂ alkyl)sulfonyl)amino or N-(phenylsulfonyl)amino. Also, the term “substituted naphthyl” represents disubstituted naphthyl groups wherein the substituents are different.

The term “naphthylene” means a naphthyl radical bonded at two positions connecting together two separate additional groups. Similarly, the term “substituted napthylene” means a naphthylene group that is further substituted by halogen, hydroxy, protected hydroxy, C₁ to C₁₀ alkylthio, C₁ to C₁₀ alkylsulfoxide, C₁ to C₁₀ alkylsulfonyl, C₁ to C₁₀ substituted alkylthio, C₁ to C₁₀ substituted alkylsulfoxide, C₁ to C₁₀ substituted alkylsulfonyl, C₁ to C₁₂ alkyl, C₁ to C₁₂ alkoxy, C₁ to C₁₂ substituted alkyl, C₁ to C₁₂ alkoxy, oxo, protected oxo, (monosubstituted)amino, (disubstituted)amino, trifluoromethyl, carboxy, protected carboxy, phenyl, substituted phenyl, phenylthio, phenylsulfoxide, phenylsulfonyl, amino, or protected amino group.

The terms “halo” and “halogen” refer to the fluoro, chloro, bromo or iodo atoms. There can be one or more halogens, which are the same or different.

The term “(monosubstituted)amino” refers to an amino group with one substituent chosen from the group consisting of phenyl, substituted phenyl, C₁ to C₁₂ alkyl, C₁ to C₁₂ substituted alkyl, C₁ to C₁₂ acyl, C₁ to C₁₂ substituted acyl, C₂ to C₁₂ alkenyl, C₂ to C₁₂ substituted alkenyl, C₂ to C₁₂ alkynyl, C₂ to C₁₂ substituted alkynyl, C₇ to C₁₈ phenylalkyl, C₇ to C₁₈ substituted phenylalkyl, heterocyclic ring, substituted heterocyclic ring, C₁ to C₁₂ heterocycloalkyl and C₁ to C₁₂ substituted heterocycloalkyl. The (monosubstituted)amino can additionally have an amino-protecting group as encompassed by the term “protected (monosubstituted)amino.”

The term “(disubstituted)amino” refers to an amino group with two substituents chosen from the group consisting of phenyl, substituted phenyl, C₁ to C₁₂ alkyl, C₁ to C₁₂ substituted alkyl, C₁ to C₁₂ acyl, C₂ to C₁₂ alkenyl, C₂ to C₁₂ alkynyl, C₇ to C₁₈ phenylalkyl, C₇ to C₁₈ substituted phenylalkyl, C₁ to C₁₂ heterocycloalkyl and C₁ to C₁₂ substituted heterocycloalkyl. The two substituents can be the same or different.

The term “amino-protecting group” as used herein refers to substituents of the amino group commonly employed to block or protect the amino functionality while reacting other functional groups of the molecule. The term “protected (monosubstituted)amino” means there is an amino-protecting group on the monosubstituted amino nitrogen atom. In addition, the term “protected carboxamide” means there is an amino-protecting group on the carboxamide nitrogen. Similarly, the term “protected N—(C₁ to C₁₂ alkyl)carboxamide” means there is an amino-protecting group on the carboxamide nitrogen.

The species of amino-protecting group employed is not critical so long as the derivatized amino group is stable to the conditions of the subsequent reaction(s) and can be removed at the appropriate point without disrupting the remainder of the compounds. Preferred amino-protecting groups are Boc, Cbz and Fmoc. Further examples of amino-protecting groups embraced by the above term are well known in organic synthesis and the peptide art and are described by, for example, T. W. Greene and P. G. M. Wuts, “Protective Groups in Organic Synthesis,” 2nd ed., John Wiley and Sons, New York, N.Y., 1991, Chapter 7, M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, each of which is incorporated herein by reference. The related term “protected amino” defines an amino group substituted with an amino-protecting group discussed above.

The term “protected guanidino” as used herein refers to an “amino-protecting group” on one or two of the guanidino nitrogen atoms. Examples of “protected guanidino” groups are described by T. W. Greene and P. G. M. Wuts; M. Bodanzsky; and Stewart and Young, supra.

The term “epimino” means —NH—. The term “substituted epimino” means —N(R)—, where R is a substitution group listed above under the definition of “(monosubstituted)amino.”

The term “C₁ to C₅ alkylene epimino” refers to a one to five carbon alkylene chain with an epimino at any point along the chain. The term “C₁ to C₅ substituted alkylene epimino” refers to a C₁ to C₅ alkylene epimino group that is substituted a) at the epimino position (in the same way as “substituted epimino,” described above); and/or b) at one or more of the alkylene positions (in the same way as “substituted alkylene,” as described above).

The term “thio” refers to —SH or, if between two other groups, —S—. The term “C₁ to C₁₀ alkylene thio” refers to a one to ten carbon alkylene chain with a thio at any point along the chain. The term “C₁ to C₁₀ substituted alkylene thio” refers to a C₁ to C₁₀ alkylene thio group that is substituted at one or more of the alkylene positions (in the same way as “substituted alkylene,” as described above).

The term “sulfonyl” refers to —S(O)₂—. The term “C₁ to C₁₀ alkylene sulfonyl” refers to a one to ten carbon alkylene chain with a sulfonyl at any point along the chain. The term “C₁ to C₁₀ substituted alkylene sulfonyl” refers to a C₁ to C₁₀ alkylene sulfonyl group that is substituted at one or more of the alkylene positions (in the same way as “substituted alkylene,” as described above).

The term “sulfinyl” refers to —S(O)—. The term “C₁ to C₁₀ alkylene sulfinyl” refers to a one to ten carbon alkylene chain with a sulfinyl at any point along the chain. The term “C₁ to C₁₀ substituted alkylene sulfinyl” refers to a C₁ to C₁₀ alkylene sulfinyl group that is substituted at one or more of the alkylene positions (in the same way as “substituted alkylene,” as described above).

The term “oxy” refers to —O—. The terms “C₁ to C₁₀ alkylene oxy,” “C₁ to C₁₀ alkylene dioxy” and “C₁ to C₁₀ alkylene trioxy” refer to a one to ten carbon alkylene chain with, respectively, one, two or three —O— at any point along the chain, provided that no two oxygen atoms are consecutive, and provided that any two oxygen atoms are separated by at least two carbons. The terms “C₁ to C₁₀ substituted alkylene oxy,” “C₁ to C₁₀ substituted alkylene dioxy” and “C₁ to C₁₀ substituted alkylene trioxy” refer, respectfully to “C₁ to C₁₀ alkylene oxy,” “C₁ to C₁₀ alkylene dioxy” and “C₁ to C₁₀ alkylene trioxy” that are substituted at one or more of the alkylene positions (in the same way as “substituted alkylene,” as described above).

The term “thiocarbonyl” refers to —C(S)H or, if between two other groups, —C(S)—. The term “thioester” refers to —C(O)SH or, if between two other groups, —C(O)S—.

The term “carboxy-protecting group” as used herein refers to one of the ester derivatives of the carboxylic acid group commonly employed to block or protect the carboxylic acid group while reactions are carried out on other functional groups on the compound. Examples of these groups are found in E. Haslam, “Protective Groups in Organic Chemistry,” J. G. W. McOmie, Ed., Plenum Press, New York, N.Y., 1973, Chapter 5, and T. W. Greene and P. G. M. Wuts, “Protective Groups in Organic Synthesis,” 2nd ed., John Wiley and Sons, New York, N.Y., 1991, Chapter 5, each of which is incorporated herein by reference. A related term is “protected carboxy,” which refers to a carboxy group substituted with one of the above carboxy-protecting groups.

The term “hydroxy-protecting group” refers to readily cleavable groups bonded to hydroxyl groups. The species of hydroxy-protecting groups is not critical so long as the derivatized hydroxyl group is stable to the conditions of subsequent reaction(s) and can be removed at the appropriate point without disrupting the remainder of the molecule. Examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2nd ed., John Wiley and Sons, New York, N.Y., 1991, Chapters 2 and 3. Related terms are “protected hydroxy,” and “protected hydroxymethyl” which refer to a hydroxy or hydroxymethyl substituted with one of the above hydroxy-protecting groups.

The term “C₁ to C₁₀ alkylthio” refers to sulfide groups such as methylthio, ethylthio, n-propylthio, isopropylthio, n-butylthio, t-butylthio and like groups.

The term “C₁ to C₁₀ alkylsulfoxide” indicates sulfoxide groups such as methylsulfoxide, ethylsulfoxide, n-propylsulfoxide, isopropylsulfoxide, n-butylsulfoxide, sec-butylsulfoxide and the like. The term “C₁ to C₁₀ alkylsulfonyl” encompasses groups such as methylsulfonyl, ethylsulfonyl, n-propylsulfonyl, isopropylsulfonyl, n-butylsulfonyl, t-butylsulfonyl and the like. it should also be understood that the above thio, sulfoxide or sulfonyl groups can be at any point on the alkyl chain (e.g., 2-methylmercaptoethyl).

The terms “C₁ to C₁₀ substituted alkylthio,” “C₁ to C₁₀ substituted alkylsulfoxide,” and “C₁ to C₁₀ substituted alkylsulfonyl,” denote the C₁ to C₁₀ alkyl portion of these groups may be substituted as described above in relation to “substituted alkyl.”

The terms “phenylthio,” “phenylsulfoxide,” and “phenylsulfonyl” specify a thiol, a sulfoxide, or sulfone, respectively, containing a phenyl group. The terms “substituted phenylthio,” “substituted phenylsulfoxide,” and “substituted phenylsulfonyl” means that the phenyl of these groups can be substituted as described above in relation to “substituted phenyl.”

The term “C₁ to C₁₂ alkylaminocarbonyl” means a C₁ to C₁₂ alkyl attached to a nitrogen of the aminocarbonyl group. Examples of C₁ to C₁₂ alkylaminocarbonyl include methylaminocarbonyl, ethylaminocarbonyl, propylaminocarbonyl and butylaminocarbonyl. The term “C₁ to C₁₂ substituted alkylaminocarbonyl” denotes a substituted alkyl bonded to a nitrogen of the aminocarbonyl group, which alkyl may be substituted as described above in relation to C₁ to C₁₂ substituted alkyl.

The term “C₁ to C₁₂ alkoxycarbonyl” means a “C₁ to C₁₂ alkoxy” group attached to a carbonyl group. The term “C₁ to C₁₂ substituted alkoxycarbonyl” denotes a substituted alkoxy bonded to the carbonyl group, which alkoxy may be substituted as described above in relation to “C₁ to C₁₂ substituted alkyl.”

The term “phenylaminocarbonyl” means a phenyl attached to a nitrogen of the aminocarbonyl group. The term “substituted phenylaminocarbonyl” denotes a substituted phenyl bonded to a nitrogen of the aminocarbonyl group, which phenyl may be substituted as described above in relation to substituted phenyl.

The term “C₁ to C₁₂ alkylaminothiocarbonyl” means a C₁ to C₁₂ alkyl attached to an aminothiocarbonyl group, wherein the alkyl has the same meaning as defined above.

The term “C₁ to C₁₂ substituted alkylaminothiocarbonyl” denotes a substituted alkyl bonded to an aminothiocarbonyl group, wherein the alkyl may be substituted as described above in relation to C₁ to C₁₂ substituted alkyl.

The term “phenylaminothiocarbonyl” means a phenyl attached to an aminothiocarbonyl group, wherein the phenyl has the same meaning as defined above.

The term “substituted phenylaminothiocarbonyl” denotes a substituted phenyl bonded to an aminothiocarbonyl group, wherein phenyl may be substituted as described above in relation to substituted phenyl.

The term “phenylene” means a phenyl group where the phenyl radical is bonded at two positions connecting together two separate additional groups.

The term “substituted phenylene” means a phenyl group where the phenyl radical is bonded at two positions connecting together two separate additional groups,

wherein the phenyl is substituted as described above in relation to “substituted phenyl.”

The term “substituted C₁ to C₁₂ alkylene” means a C₁ to C₁₂ alkyl group where the alkyl radical is bonded at two positions connecting together two separate additional groups and further bearing an additional substituent.

The terms “cyclic C₂ to C₇ alkylene,” “substituted cyclic C₂ to C₇ alkylene,” “cyclic C₂ to C₇ heteroalkylene,” and “substituted cyclic C₂ to C₇ heteroalkylene,” defines such a cyclic group bonded (“fused”) to the phenyl radical resulting in a bicyclic ring system. The cyclic group may be saturated or contain one or two double bonds. Furthermore, the cyclic group may have one or two methylene or methine groups replaced by one or two oxygen, nitrogen or sulfur atoms that are the cyclic C₂ to C₇ heteroalkylene.

The cyclic alkylene or heteroalkylene group may be substituted once or twice by the same or different substituents which, if appropriate, can be connected to another part of the compound (e.g., alkylene) selected from the group consisting of the following moieties: hydroxy, protected hydroxy, carboxy, protected carboxy, oxo, protected oxo, C₁ to C₄ acyloxy, formyl, C₁ to C₁₂ acyl, C₁ to C₁₂ alkyl, C₁ to C₇ alkoxy, C₁ to C₁₀ alkylthio, C₁ to C₁₀ alkylsulfoxide, C₁ to C₁₀ alkylsulfonyl, halo, amino, protected amino, (monosubstituted)amino, protected (monosubstituted)amino, (disubstituted)amino, hydroxymethyl or a protected hydroxymethyl.

The cyclic alkylene or heteroalkylene group fused onto the benzene radical can contain two to ten ring members, but it preferably contains three to six members. Examples of such saturated cyclic groups are when the resultant bicyclic ring system is 2,3-dihydro-indanyl and a tetralin ring. When the cyclic groups are unsaturated, examples occur when the resultant bicyclic ring system is a naphthyl ring or indolyl. Examples of fused cyclic groups which each contain one nitrogen atom and one or more double bond, preferably one or two double bonds, are when the benzene radical is fused to a pyridino, pyrano, pyrrolo, pyridinyl, dihydropyrrolo, or dihydropyridinyl ring. Examples of fused cyclic groups which each contain one oxygen atom and one or two double bonds are when the benzene radical ring is fused to a furo, pyrano, dihydrofurano, or dihydropyrano ring. Examples of fused cyclic groups which each have one sulfur atom and contain one or two double bonds are when the benzene radical is fused to a thieno, thiopyrano, dihydrothieno or dihydrothiopyrano ring. Examples of cyclic groups that contain two heteroatoms selected from sulfur and nitrogen and one or two double bonds are when the benzene radical ring is fused to a thiazolo, isothiazolo, dihydrothiazolo or dihydroisothiazolo ring. Examples of cyclic groups which contain two heteroatoms selected from oxygen and nitrogen and one or two double bonds are when the benzene ring is fused to an oxazolo, isoxazolo, dihydrooxazolo or dihydroisoxazolo ring. Examples of cyclic groups which contain two nitrogen heteroatoms and one or two double bonds occur when the benzene ring is fused to a pyrazolo, imidazolo, dihydropyrazolo or dihydroimidazolo ring or pyrazinyl.

The term “carbamoyl” means an —NC(O)— group where the radical is bonded at two positions connecting two separate additional groups.

The term “organic or inorganic cation” refers to counter-ions for the carboxylate anion of a carboxylate salt. The counter-ions are chosen from the alkali and alkaline earth metals, (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, “Pharmaceutical Salts,” Berge et al., J. Pharm. Sci., 66:1-19 (1977), which is incorporated herein by reference. Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group is referred to by this term. For example, a cation for a carboxylate anion will exist when a position is substituted with a (quaternary ammonium)methyl group. A preferred cation for the carboxylate anion is the sodium cation.

The compounds of the invention can also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

One or more compounds of the invention can be in the biologically active ester form, such as the non-toxic, metabolically-labile ester-form. Such ester forms induce increased blood levels and prolong the efficacy of the corresponding non-esterified forms of the compounds. Ester groups which can be used include the lower alkoxymethyl groups, for example, methoxymethyl, ethoxymethyl, isopropoxymethyl and the like; the —(C₁ to C₁₂) alkoxyethyl groups, for example methoxyethyl, ethoxyethyl, propoxyethyl, isopropoxyethyl and the like; the 2-oxo-1,3-diooxlen-4-ylmethyl groups, such as 5-methyl-2-oxo-1,3-dioxolen-4-ylmethyl, 5-phenyl-2-oxo-1,3-dioxolen-4-ylmethyl and the like; the C₁ to C₁₀ alkylthiomethyl groups, for example methylthiomethyl, ethylthiomethyl, iso-propylthiomethyl and the like; the acyloxymethyl groups, for example pivaloyloxymethyl, pivaloyloxyethyl, -acetoxymethyl and the like; the ethoxycarbonyl-1-methyl group; the -acetoxyethyl; the 1-(C₁ to C₁₂ alkyloxycarbonyloxy)ethyl groups such as the 1-(ethoxycarbonyloxy)ethyl group; and the 1-(C₁ to C₁₂ alkylaminocarbonyloxy)ethyl groups such as the 1-(methylaminocarbonyloxy)ethyl group.

The term “amino acid” includes any one of the twenty naturally-occurring amino acids or the D-form of any one of the naturally-occurring amino acids. In addition, the term “amino acid” also includes other non-naturally occurring amino acids besides the D-amino acids, which are functional equivalents of the naturally-occurring amino acids. Such non-naturally-occurring amino acids include, for example, norleucine (“Nle”), norvaline (“Nva”), L- or D-naphthalanine, ornithine (“Orn”), homoarginine (homoArg) and others well known in the peptide art, such as those described in M. Bodanzsky, “Principles of Peptide Synthesis,” 1st and 2nd revised ed., Springer-Verlag, New York, N.Y., 1984 and 1993, and Stewart and Young, “Solid Phase Peptide Synthesis,” 2nd ed., Pierce Chemical Co., Rockford, Ill., 1984, both of which are incorporated herein by reference. Amino acids and amino acid analogs can be purchased commercially (Sigma Chemical Co.; Advanced Chemtech) or synthesized using methods known in the art.

It should be understood that any position of the claimed invention has up to three serial “substitutions.” For example, a “substituted alkyl” that is substituted with a “substituted phenyl” that is, in turn, substituted with a “substituted alkyl” can, in turn, be substituted by one more group and no longer further substituted. However, it should also be understood that the invention contemplates, if appropriate, more than three parallel substitutions. For example, if appropriate, more than three hydrogens on an alkyl moiety may be substituted with any one or more of a variety of groups, including halo and hydroxy.

### General Synthesis of Quinolinium Derivatives

Synthesis of example compounds was accomplished as shown in Scheme 1. Alkylation of various quinolines was accomplished using alkyl triflates, alkyl sulfates, alkyl methnaesulfonates or alkyl tosylates in dichloromethane, chloroform or toluene to afford the desired quinolinium salts. Purification consisted of evaporation to dryness, or a simple filtration and yields were consistently high. N-substituted pyrroles were synthesized by condensation of a primary amine with 2,5-hexadione using catalytic iodine, or catalytic acetic acid. The pyrroles were formylated Using Vilsmeier-Haack conditions. A piperidine catalyzed condensation between the aldehyde and quinolinium salt provided the desired final quinolinium derivatives.

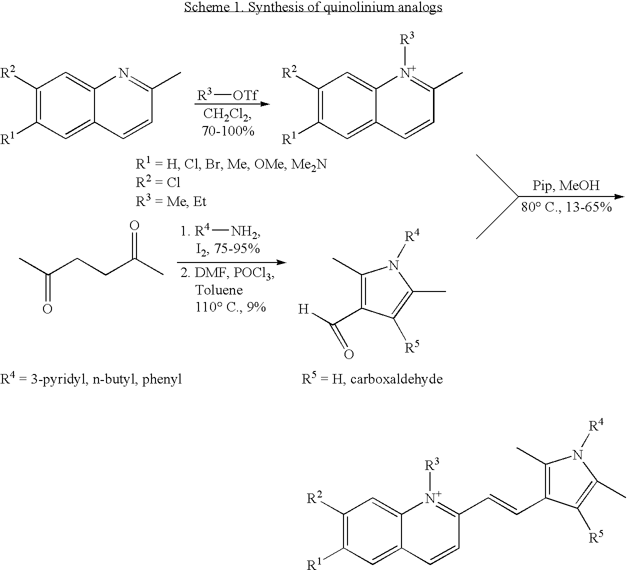

General procedure for alkylation of quinaldine to provide intermediates A. (Tetrahedron 2003, 59, 4911-4921):

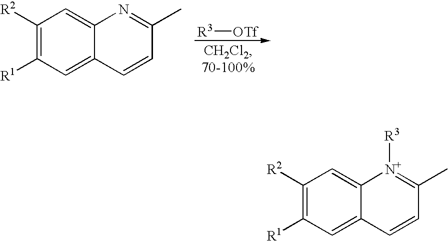

General procedure for formation of pyrrole to provide intermediates B. (J. Org. Chem. 2004, 69, 213-216):

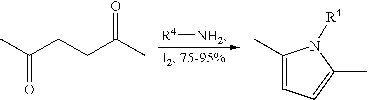

General Procedure for formylation to form intermediates C. (J. Org. Chem. 1959, 24, 372-374):

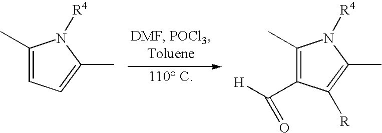

### General Procedure for Condensation:

To a solution of the 2-methylquinolinium salt (0.571 mmol) and aldehyde (0.571 mmol) in methanol (1 mL) was added piperidine (1-2 drops) and refluxed for 4-16 h. The solution was cooled to room temperature and the precipitate collected using vacuum filtration to afford a solid.

### EXAMPLES

### Preparation of Intermediates

Intermediate 1: (2,5-dimethyl-1-phenyl-1H-pyrrole-3,4-dicarboxaldehyde) was made as follows:

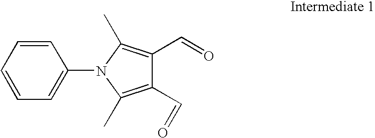

To a well stirred solution of 2,5-dimethyl-1-phenylpyrrole (Banik J. Org. Chem. 2004, 69, 213-216) (12 mmol) and DMF (10.2 eq) in anhydrous toluene (9 mL) was added POCl₃ (8.2 eq) dropwise. The solution was heated to 110° C. for 6 h. The mixture was cooled to room temperature. NaOAc(sat) (100 mL) was added and then vigorously stirred for 20 min. Dichloromethane (500 mL) and 5 g of K₂CO₃(s) were added. The mixture was shaken until all color disappeared from the aqueous layer. The organic layer was washed with NaHCO₃ and brine and evaporated to provide 2,5-dimethyl-1-phenyl-1H-pyrrole-3,4-dicarboxaldehyde.

¹HNMR (CDCl₃): δ 10.29 (s, 2H), 3.83 (t, J=7.8 Hz, 2H), 2.54 (s, 6H), 1.66-1.62 (m, 2H), 1.43 (m, 2H), 0.98 (t, J=7.4 Hz, 3H).

Intermediate 2: (4′-Formyl-biphenyl-3-carboxylic acid methyl ester) was made as follows:

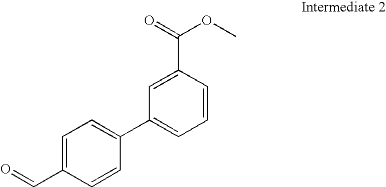

To a solution of 4′-Formyl-biphenyl-3-carboxylic acid (0.500 g, 2.21 mmol) and potassium carbonate (0.305 g, 2.21 mmol) in dichloromethane (10 mL) was added methyl triflate (0.26 mL, 2.32 mmol) dropwise. The solution was stirred at room temperature for 16 h. The ppt was filtered and the organic layer concentrated to afford a white solid (0.395 g, 74%). ¹H NMR (CDCl₃) 10 1 (s, 1H) 8.33 (t, J=1.8 Hz, 1H), 8.09 (dd, J=9.1 Hz, 1H), 7.99-7.97 (m, 2H), 7.84-7.80 (m, 1H), 7.80 (dd, J=6.6, 1.8 Hz, 2H), 7.57 (t, J=7.9 Hz, 1H), 3.97 (s, 3H).

Intermediate 3:

### 1,2-dimethylquinolinium Triflate

To a stirred solution of quinaldine (1.34 mmol) in dichloromethane (7 mL) at room temperature was added methyl triflate, (1.47 mmol) dropwise. The solution was stirred for 16 h resulting in a precipitate that was collected via vacuum filtration to provide 1,2-dimethylquinolinium Triflate

¹H NMR (DMSO) d 8.71 (d, J=8.5 Hz, 1H), 8.34 (d, J=9.8 Hz, 1H), 7.85 (d, J=8.6 Hz, 1H), 7.76 (d, J=9.7, 3.0 Hz, 1H), 7.28-7.25 (m, 1H), 4.34 (s, 3H), 3.77 (s, 3H), 3.11 (s, 6H).

Intermediate 4:

### 6-bromo-1,2-dimethylquinolinium Triflate

Using the method of intermediate 3

¹H NMR (DMSO) d 8.99 (d, J=8.6 Hz, 1H), 8.71 (d, J=2.3 Hz, 1H), 8.53 (d, J=9.5 Hz, 1H), 8.36 (dd, J=9.5, 2.4 Hz, 1H), 8.15 (d, J=8.6 Hz, 2H), 3.03 (s, 3H), 3.05 (s, 3H).

HPLC was used to characterize the remaining compounds. A Zorbax-SB C-18 analytical column (5 mm) was used with a flow rate of 1.5 mL/min. with the eluent 0.05% TFA in H₂O to 0.05% TFA in CH₃CN (0-100% over 15 min) was used.

TABLE 2
Retention times (Rₜ) of quinolinium salts

| | | | | | Alkylating |
|---|---|---|---|---|---|
| Intermediate | R¹ | R² | R³ | Rₜ | Reagent |
| 3 | H | H | Me | 4.2 | MeOTf |
| 4 | Cl | H | Me | 5.1 | MeOTf |
| 5 | Br | H | Me | 5.4 | MeOTf |
| 6 | Ome | H | Me | 6.5 | MeOTf |
| 7 | Me₂N | H | Me | 6.1 | MeOTf |
| | | | | | MeSO3Me |
| | | | | | MeOSO2Tos |
| 8 | H | Cl | Me | 4.9 | MeOTf |
| | | | | | MeSO3Me |
| 9 | Br | H | Et | 5.9 | EtOTf |
| 10 | Me₂N | H | Et | 6.4 | EtOTf, Et₂SO4 |
| 11 | Me | H | Me | | MeSO3Me |
| | | | | | MeOSO2Tos |

Intermediate 12:

### 2,5-dimethyl-1-phenylpyrrole

To a stirred solution of 2,5-hexadione (1.2 mmol) and aniline (1 mmol) at room temperature was added iodine (0.1 mmol). The solution was stirred for 16 h. The solution was diluted with dichloromethane (20 mL) and then washed successively with 5% NaSSO₃, NaHCO₃ and brine (5 mL each). The organic layer was dried with MgSO₄ and concentrated under vacuum to afford the 2,5-dimethyl-1-phenylpyrrole

¹HNMR agrees with lit values (J. Org. Chem. 1959, 24, 372-374).

Intermediate 13:

### 2,5-dimethyl-1-(3-pyridyl)pyrrole

Method as for Intermediate 12

¹HNMR (CDCl₃): δ 8.60 (dd, J=4.7, 1.7 Hz, 1H), 7.81 (td, J=7.8, 1.9 Hz, 1H), 7.29 (dd, J=7.2, 5.0 Hz, 1H), 7.22 (d, J=7.9 Hz, 1H), 5.89 (s, 2H), 2.18 (s, 6H).

Intermediate 14:

### 1-butyl-2,5-dimethylpyrrole

Method as for Intermediate 12

¹HNMR (CDCl₃): δ 5.76 (s, 2H), 3.72 (t, J=3.6 Hz, 2H), 2.22 (s, 6H), 1.63-1.57 (m, 2H), 1.39-1.35 (m, 2H), 0.96 (t, J=7.4 Hz, 3H).

Alternate Method for Intermediate 14:

A sample of butylamine, 6.3 grams, was treated with 1,5-hexanedione, 9.6 grams, and then 2 ul of acetic acid was added. The mixture was warmed to the touch, and placed in an ice bath. After four hours the ice bath had melted, and 3 ml of a water layer had separated. The mix was treated with 30 ml hexanes and 10 ml of water, and stored at −20° C. The next day the organic layer was decanted from the frozen aqueous layer, then the organic layer was evaporated in vacuo to provide 12.24 grams of 1-butyl-2,5-dimethylpyrrole as a light yellow oil.

Intermediate 15:

### 2,5-dimethyl-1-phenyl-1H-pyrrole-3-carbaldehyde

To a well stirred solution of 2,5-dimethyl-1-phenylpyrrole (12 mmol) and DMF (12 mmol) in anhydrous toluene (9 mL) was added POCl₃ (12 mmol) dropwise. The solution was heated to 110° C. for 6 h. The mixture was cooled to room temperature. NaOAc(sat) (100 mL) was added and then vigorously stirred for 20 min. Dichloromethane (500 mL) and 5 g of K₂CO₃(s) were added. The mixture was shaken until all color disappeared from the aqueous layer. The organic layer was washed with NaHCO₃ and brine and evaporated to provide-2,5-dimethyl-1-phenyl-1H-pyrrole-3-carbaldehyde.

¹HNMR (CDCl₃): δ 9.87 (s, 1H), 7.54-7.48 (m, 3H), 7.21-7.19 (m, 2H), 6.38 (s, 1H), 2.27 (s, 3H), 1.98 (s, 3H).

Intermediate 16:

### 2,5-dimethyl-1-pyridin-3-yl-1H-pyrrole-3-carbaldehyde

Method as for Intermediate 15

¹HNMR (CDCl₃): δ 9.89 (s, 1H), 8.75-8.74 (m, 1H), 8.54 (m, 1H), 7.61-7.58 (m, 1H), 7.52-7.49 (m, 1H), 6.42 (s, 1 h), 2.30 (s, 3H), 2.00 (s, 3H).

Intermediate 17:

### 2,5-dimethyl-1-butyl-1H-pyrrole-3-carbaldehyde

Prepared as for intermediate 15, TLC on Silica, Rf=0.3 using 1:1 EtOAc and hexanes.

¹H NMR (CDCl₃) δ 10.30 (s, 2H), 3.83-3.80 (m, 2H), 3.15 (s, 6H), 1.65-1.62 (m, 2H), 1.43-1.38 (m, 2H), 0.99 (t, J=7.4, 3H).

Intermediate 18:

### 4-benzyloxy-3-chlorobenzaldehyde

A sample of 3-chloro-4-hydroxybenzaldehyde, 0.156 g, was placed in a 13×100 mm test tube with stirring bar. Then anhydrous potassium carbonate, 0.33 g, was added, followed by 0.5 ml of dry acetone. Then 0.12 ml of benzyl bromide was added, and the mixture stirred at room temperature over night. After stirring 22 hours, 2 ml of dichloromethane was added, and the solids removed by filtration. TLC of the filtrate showed a new spot on TLC with dichloromethane on silica, RF 0.7, and no stating material RF 0.2. Evaporation gave a white sold.

Intermediate 19:

### 3,4-diformyl-2,5-dimethyl-1-(2-phenylethyl)pyrrole

A: 2,5-dimethyl-1-(2-phenylethyl)pyrrole

A sample of 1.67 g of hexan-2,5-dione was place in a 20 ml vial with a stirring bar. Then 1.67 ml of phenethylamine was added, followed by 0.1 ml of acetic acid. On addition of the acetic acid, the mixture rapidly warmed, and be came cloudy. The mixture was stirred 2 hours, then 10 ml of hexanes and 2 ml of water was added. The mix was stirred overnight. The organic layer was separated, and evaporated to give 2.5 grams of light yellow oil that became a solid on storage at −20° C.

NMR DMSO 7.27-7.11, M, 5H, 5.58, S, 2H, 3.90, T, 2H, 2.80, T, 2H

B: 3,4-diformyl-2,5-dimethyl-1-(2-phenylethyl)pyrrole 2,5-dimethyl-1-(2-phenylethyl)pyrrole, 1.0 g, was placed in a 20×150 ml test tube with a stirring bar, and 5 ml of dry DMF was added. The mixture was the treated with 1.8 ml of POCl₃ dropwise over 5 minutes. The mixture exothermically warmed to 85° C., and was the heated at 100 C for 1 hour. TLC on silica with 10% acetone in CH₂Cl₂ showed a new spot RF 0.5, and no starting material, RF 0.8. The reaction was poured onto 90 g of ice and 8 g of sodium acetate. The mix was treated with 30 ml of ethyl acetate, and stirred vigorously for 5 minutes. The mix was then seperated, and the top layer was dried with magnesium sulfate, and then evaporated in vacuo to provide a dark oil. The oil was chromatographed on silica, 1×2.5 inch column. The oil was dissolved in 40 ml of dichloromethane, and adsorbed on the column. The column was then eluted with 2×40 ml of 5% ethyl acetate in dichloromethane. Fractions judged to be pure by TLC were combined and evaporated in vacuo to give a yellow oil that solidified on standing.

NMR DMSO 10.16, S, 2H, 7.31-7.24, M, 3H, 7.15-7.14, M, 2H, 4.14, T, 2H, 2.93, T, 2H, 2.37, S, 6H

Intermediate 20

### 1-butyl-3,4-diformyl-2,5-dimethylpyrrole

In a 250 ml round bottom flask with magnetic stirring bar and thermometer was placed intermediate 14, 12.24 grams. Then dimethylformamide, 60 ml, was added. The flask was cooled in an ice bath and the mixture was then treated with phosphorus oxychloride, 19 ml, in 1 ml portions. For the first 8 ml, a vigorous exotherm warmed the solution from 10 C to 20 C with each 1 ml portion. After the first 8 ml, additional phosphorus oxychloride resulted in little warming. After the addition of the phosphorus oxychloride, the ice bath was removed, and the flask containing the light orange solution was placed in an oil bath, and then the bath was heated to 100 C. The solution reached 90 C after 45 minutes, and cooled to 85 C over the next 45 minutes. After heating two hours, the oil bath was removed and the solution was allowed to cool for 15 minutes. The solution was them poured onto a mixture of 600 grams of ice and 100 grams of sodium acetate, and stirred. After 25 minutes a suspension of gray solids had formed, the mixture was filtered, and the solids were washed with 2×100 ml water, and air dried to provide 1-butyl-3,4-diformyl-2,5-dimethylpyrrole, 9.8 g. Solids show one spot, RF 0.3, on silica TLC with 10% ethyl acetate in dichloromethane.

¹HNMR (DMSO, 500 MH): 10.18 (s, 2H), 3.90 (dd, 2H), 2.51 (s, 6H), 1.56 (m, 2H), 1.34 (m, 2H), 0.92 (t, 3H)

Intermediate 21

### 1,2-dimethylquinolinium tosylate

To a solution of 6-dimethylaminoquinaldine (5.0 g, 26.8 mmol) in 4 mL of CHCl₃ was added 4.0 mL of methane p-toluenesulphonate and heated to reflux for 12 h. The reaction was concentrated to give the desired product.

¹H NMR (DMSO-d₆) δ 8.67 (d, J=9.2, 1H), 8.28 (d, J=9.8, 1H), 7.80 (d, J=8.7, 1H), 7.69-7.66 (m, 1H), 7.48 (d, J=8.0, 2H), 4.31 (s, 3H), 3.07 (s, 6H), 2.98 (s, 3H), 2.25 (s, 3H).

### Example 1

### Synthesis of Pyrvinium Phosphate

A sample of pyrvinium pamoate, 0.51 g, ICN # 156475, lot 8256B, was placed in a 250 ml Erlenmeyer flask with a magnetic stirring bar. Then 40 ml of chloroform, Fischer BP1145-1, lot 982393, was added, followed by 20 ml of 95% ethanol, Phamaco ACS/USP grade lot 0204092 batch 02102-15, giving a deep red solution.

The mixture was warmed to 50° C. and stirred 10 minutes. The mixture was then treated with 10 ml of 2% phosphoric acid (85%) in 95% ethanol, producing a precipitate. After 2 minutes, 30 ml of ethyl acetate was added, and the mixture was stirred for 20 minutes. The solids were then collected by filtration, washed with 20 ml of 2/1/1 ethyl acetate/chloroform/ethanol, and air dried to provide 0.45 g of brick red powder. CNH found av 2 runs C 50.75H 6.11 N 6.70, calc for C26H28N3.H2PO4.H3PO4.2H2O C 50.90H 6.08 N 6.85

A sample of the Example 1 phosphate salt was freely soluble in water at 1 mg/ml, giving an orange red solution. This contrasts the poor solubility of pyrvinium pamoate, which when stirred in water at 1 mg/ml for 16 hours at RT and then filtered gave a colorless solution with pyrvinium below the limit of detection by HPLC analysis.

### Example 2

### Synthesis of Pyrvinium Sulfate

A sample of 0.51 g of pyrvinium pamoate was placed in a 250 ml Erlenmeyer flask, and a 1.5×⅜ inch string bar was added. Then 40 ml of Chloroform (ethanol stablised) and 20 ml of 95% ethanol were added, and the mixture was stirred, giving a deep red solution. After 10 minutes a solution of 0.11 ml of 95% sulfuric acid in 4 ml of 95% ethanol was added dropwise, resulting in a deep red solution. After stirring 5 minutes, 50 ml of Ethyl Acetate was poured in as one portion. The resulting suspension of solids was stirred 20 minutes, and then filtered to collect solids. The solids were washed with 2×5 ml of ethyl acetate, and air dried to give 0.4035 g of fine powdery deep red solids. CHN Found C, 61.09; H, 6.08; N, 8.07; S, 6.53; KF, 5.40 Calc C26H28N3+.HSO4−.4H20 61.656.378.30, S 6.32 KF 5.33

The pyrvinium sulfate is soluble to 1 mg/ml in 1.5% DMSO and 5% dextrose in water.

### Example 3

### Synthesis of 6-dimethylamino-2-[(E)-2-(4-dimethylamino-phenyl)-vinyl]-1-methyl-quinolinium triflate

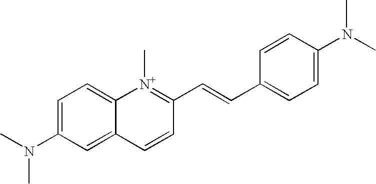

To a solution of 6-dimethylamino-1,2-dimethylquinolinium triflate (0.200 g, 0.571 mmol) and 4-(dimethylamino)benzaldehyde (85 mg, 0.571 mmol) in MeOH (1 mL) was added piperidine (1-2 drops) and refluxed for 4 h. The solution was cooled to room temperature and the precipitate collected using vacuum filtration to afford a purple solid (102 mg, 37%). ¹H NMR (DMSO-d₆) δ 8.55 (d, J=9.4 Hz, 1H), 8.31 (d, J=9.3 Hz, 1H), 8.25 (d, J=9.8 Hz, 1H), 8.01 (d, J=15.6 Hz, 1H), 7.78 (d, J=9.0 Hz, 2H), 7.63 (dd, J=9.8, 3.0 Hz, 1H), 7.50 (d, J=15.6 Hz, 1H), 7.23 (d, J=3.0 Hz, 1H), 6.81 (d, J=9.0 Hz, 2H), 4.40 (s, 3H), 3.11 (s, 6H), 3.06 (s, 6H); HPLC Rₜ=10.0.

### Example 4

### Synthesis of 6-Dimethylamino-2-[(E)-2-(4-methoxy-phenyl)-vinyl]-1-methyl-quinolinium triflate

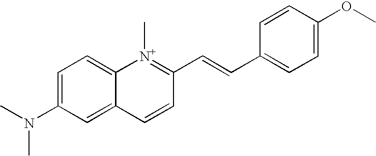

To a solution of 6-dimethylamino-1,2-dimethylquinolinium triflate (0.200 g, 0.571 mmol) and p-anisaldehyde (69 uL, 0.571 mmol) in MeOH (1 mL) was added piperidine (1-2 drops) and refluxed for 4 h. The solution was cooled to room temperature and the precipitate collected using vacuum filtration to afford a reddish-purple solid (121 mg, 45%).¹H NMR (DMSO-d₆) δ 8.67 (d, J=9.1 Hz, 1H), 8.34-8.31 (m, 2H), 7.99 (d, J=15.9 Hz, 1H), 7.89 (d, J=8.8 Hz, 2H), 7.72-7.69 (m, 2H), 7.26 (d, J=3.0 Hz, 1H), 7.09 (d, J=7.0 Hz, 2H), 4.47 (s, 3H), 3.20 (s, 3H), 3.13 (s, 6H); HPLC Rₜ=10.0.

### Example 5

### Synthesis of 2-((E)-2-Biphenyl-4-yl-vinyl)-6-dimethylamino-1-methyl-quinolinium triflate

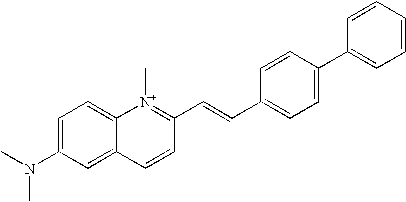

To a solution of 6-dimethylamino-1,2-dimethylquinolinium triflate (0.200 g, 0.571 mmol) and 4-biphenylcarboxaldehyde (104 mg, 0.571 mmol) in MeOH (1 mL) was added piperidine (1-2 drops) and refluxed for 4 hours. The solution was cooled to room temperature and the precipitate collected using vacuum filtration to afford a red solid (150 mg, 51%). ¹HNMR (DMSO-d₆) δ 8.73 (d, J=9.1 Hz, 1H), 8.39-8.35 (m, 2H), 8.08-7.90 (m, 3H), 7.92 (d, J=15.9 Hz, 1H), 7.85 (d, J=8.4 Hz, 2H), 7.78 (dd, J=7.4, 1.8 Hz, 2H), 7.74 (dd, J=9.3, 3.0 Hz, 1H), 7.52 (t, J=7.4 Hz, 2H), 7.43 (td, J=7.1, 1.2 Hz, 1H), 7.28 (d, J=3.1 Hz, 1H), 4.53 (s, 3H), 3.15 (s, 6H); HPLC Rₜ=11.7

### Example 6

### Synthesis of 6-dimethylamino-1-methyl-2-[(E)-2-(5-(4-methylphenyl)-isoxazol-3-yl)-vinyl]-quinolinium triflate

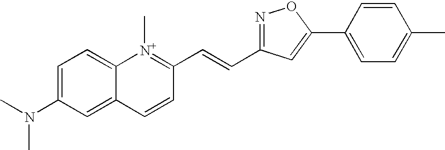

To a solution of 6-dimethylamino-1,2-dimethylquinolinium triflate (0.200 g, 0.571 mmol) and 5-(4-methylphenyl)-isoxazole-3-carboxaldehyde (99 mg, 0.571 mmol) in MeOH (1 mL) was added piperidine (1-2 drops) and refluxed for 4 h. The solution was cooled to room temperature and the precipitate collected using vacuum filtration to afford a red solid (126 mg, 43%). ¹H NMR (DMSO-d₆) δ 8.78 (d, J=9.1 Hz, 1H), 8.44 (d, J=9.0 Hz, 1H), 8.41 (d, J=10.0 Hz, 1H), 8.04 (d, J=16.0 Hz, 1H), 7.95 (d, J=16.0 Hz, 1H), 7.83-7.81 (m, 3H), 7.76 (s, 1H) 7.42 (d, J=8.0 Hz, 2H), 7.30 (d, J=3.0 Hz, 1H), 4.54 (s, 3H), 3.17 (s, 6H), 2.40 (s, 3H); HPLC Rₜ=11.2.

### Example 7

### Synthesis of 6-dimethylamino-1-methyl-2-[(E)-2-(4-phenyl-thiophen-2-yl)-vinyl]-quinolinium triflate

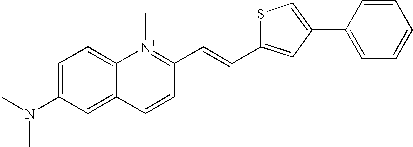

To a solution of 6-dimethylamino-1,2-dimethylquinolinium triflate (0.200 g, 0.571 mmol) and 4-phenyl-thiophene-2-carboxaldehyde (107 mg, 0.571 mmol) in MeOH (1 mL) was added piperidine (1-2 drops) and refluxed for 4 h. The solution was cooled to room temperature and the precipitate collected using vacuum filtration to afford a red solid (142 mg, 48%). ¹H NMR (DMSO-d₆) δ 8.69 (d, J=9.2, 1H), 8.34 (d, J=9.9 Hz, 1H), 8.32 (d, J=9.2 Hz, 1H), 8.23 (s, 1H), 8.21 (s, 1H), 8.19 (d, J=15.7 Hz, 1H), 7.78 (dd, J=8.4, 1.2 Hz, 2H), 7.73 (dd, J=9.8, 3.0 Hz, 1H), 7.66 (d, J=15.6, 1H), 7.48 (td, J=9.2, 2H), 7.38-7.35 (m, 1H), 7.26 (d, J=3.0, 1H), 4.48 (s, 3H), 3.14 (s, 6H); HPLC Rₜ=11.7.

### Example 8

### Synthesis of 6-dimethylamino-2-[(E)-2-(1H-indol-3-yl)-vinyl]-1-methyl-quinolinium triflate

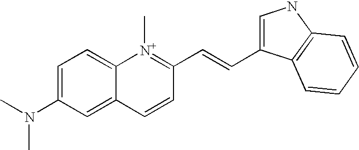

To a solution of 6-dimethylamino-1,2-dimethylquinolinium triflate (0.200 g, 0. mmol) and indole-3-carboxaldehyde (83 mg, 0.571 mmol) in MeOH (1 mL) was added piperidine (1-2 drops) and refluxed for 4 h. The solution was cooled to room temperature and the precipitate collected using vacuum filtration to afford a dark red solid (95 mg, 35%). ¹H NMR (DMSO-d₆) δ 12.1 (s, 1H), 8.57 (d, J=9.2 Hz, 1H), 8.40 (d, J=9.2 Hz, 1H), 8.38 (d, J=15.6 Hz, 1H), 8.27 (d, J=9.3 Hz, 1H), 8.24 (s, 1H) 8.18-8.16 (m, 1H), 7.63 (dd, J=9.8, 3.0 Hz, 1H), 7.53-7.50 (m, 2H), 7.29-7.24 (m, 3H), 4.42 (s, 3H), 3.12 (s, 6H); HPLC Rₜ=10.0

### Example 9

### Synthesis of 6-dimethylamino-1-methyl-2-[(E)-2-(5-phenyl-furan-2-yl)-vinyl]-quinoliNium triflate

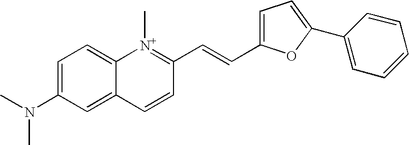

To a solution of 6-dimethylamino-1,2-dimethylquinolinium triflate (0.200 g, 0.571 mmol) and 5-phenyl-2-furaldehyde (98 mg, 0.571 mmol) in MeOH (1 mL) was added piperidine (1-2 drops) and refluxed for 4 h. The solution was cooled to room temperature and the precipitate collected using vacuum filtration to afford a red solid (142 mg, 49%). ¹H NMR (DMSO-d₆) δ 8.65 (d, J=9.1 Hz, 1H), 8.35 (d, J=9.8 Hz, 1H), 8.32 (d, J=9.2 Hz, 1H), 7.99-7.97 (m, 2H), 7.95 (d, J=15.6 Hz, 1H), 7.71 (dd, J=9.8, 3.0 Hz, 1H), 7.65 (d, J=15.6 Hz, 1H), 7.52 (t, J=7.6 Hz, 2H), 7.44-7.40 (m, 1H), 7.29 (d, J=3.8 Hz, 1H), 7.26 (d, J=3.0 Hz, 1H), 7.21 (d, J=3.6 Hz, 1H), 4.49 (s, 3H), 3.14 (s, 6H); HPLC Rₜ=11.4

### Example 10

### Synthesis of 6-dimethylamino-2-[(E)-2-(4-formyl-2,5-dimethyl-1-phenyl-1H-pyrrol-3-yl)-vinyl]-1-methyl-quinolinium triflate

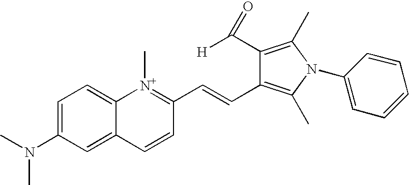

To a solution of A 6-dimethylamino-1,2-dimethylquinolinium triflate (0.111 g, 0.318 mmol) and 2,5-dimethyl-1-phenyl-1H-pyrrole-3,4-dicarboxaldehyde (132 mg, 0.636 mmol) in MeOH (1 mL) was added piperidine (1-2 drops) and refluxed for 4 h. The solution was cooled to room temperature and the precipitate collected using vacuum filtration to afford a red solid (49 mg, 29%). ¹H NMR (DMSO-d₆) δ 10.0 (s, 1H), 8.60 (d, J=9.2 Hz, 1H), 8.42 (d, J=9.3 Hz, 1H), 8.34 (d, J=15.4 Hz, 1H), 8.25 (d, J=9.8 Hz, 1H), 7.99 (d, J=15.4 Hz, 1H), 7.66 (dd, J=9.8, 3.1 Hz, 1H), 7.25 (d, J=3.0 Hz, 1H), 4.40 (s, 3H), 3.98 (t, J=7.8 Hz, 2H), 3.12 (s, 6H), 2.59 (s, 3H), 2.53 (s, 3H), 1.62-1.59 (m, 2H), 1.39-1.34 (m, 2H), 0.95 (t, J=7.4 Hz, 3H).

### Example 11

### Synthesis of 6-dimethylamino-2-[(E)-2-(3′-methoxycarbonyl-biphenyl-4-yl)-vinyl]-1-methyl-quinolinium triflate

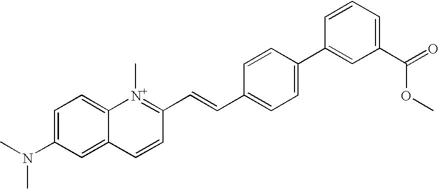

To a solution of A 6-dimethylamino-1,2-dimethylquinolinium triflate (0.200 g, 0.571 mmol) and intermediate 2 (mg, 0.571 mmol) in MeOH (1 mL) was added piperidine (1-2 drops) and refluxed for 4 h The solution was cooled to room temperature and the precipitate collected using vacuum filtration to afford a color solid (mg, %). ¹H NMR (DMSO-d₆) δ 8.72 (d, J=9.0 Hz, 1H), 8.37-8.27 (m, 2H), 8.27-8.26 (m, 1H), 8.07-8.01 (m, 5H), 7.93-7.83 (m, 3H), 7.73 (dd, J=9.8, 3.1 Hz, 1H), 7.68 (t, J=7.8 Hz, 1H), 7.26 (d, J=3.0 Hz, 1H), 4.52 (s, 3H), 3.91 (s, 3H), 3.14 (s, 3H); HPLC Rₜ=11.4

### Example 12

### Synthesis of 6-dimethylamino-2-[(E)-2-(2,5-dimethyl-1-pyridin-3-yl-1H-pyrrol-3-yl)-vinyl]-1-methyl-quinolinium triflate

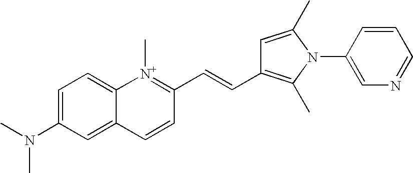

To a solution of 6-dimethylamino-1,2-dimethylquinolinium triflate (0.200 g, 0.571 mmol) and 2,5-dimethyl-1-pyridin-3-yl-1H-pyrrole-3-carbaldehyde (114 mg, 0.571 mmol) in MeOH (1.3 mL) was added piperidine (1-2 drops) and refluxed for 4 h. The solution was cooled to room temperature and the precipitate collected using vacuum filtration to afford a red solid (131 mg, 43%). ¹H NMR (DMSO-d₆) δ 8.73 (dd, J=4.8, 1.5 Hz, 1H), 8.63 (d, J=2.4 Hz, 1H), 8.54 (d, J=9.2 Hz, 1H), 8.46 (d, J=9.3 Hz, 1H), 8.24 (d, J=9.7 Hz, 1H), 8.09 (d, J=15.2 Hz, 1H), 7.92-7.90 (m, 1H), 7.65-7.60 (m, 2H), 7.27 (d, J=15.2 Hz, 1H), 7.23 (d, J=3.0 Hz, 1H), 6.78 (s, 1H), 4.37 (s, 3H), 3.11 (s, 6H), 2.26 (s, 3H), 2.03 (s, 3H); HPLC Rₜ=9.3

### Example 13

### Synthesis of 6-dimethylamino-2-[(E)-2-(2,5-dimethyl-1-phenyl-1H-pyrrol-3-yl)-vinyl]-1-ethyl-quinolinium ethylsulfate

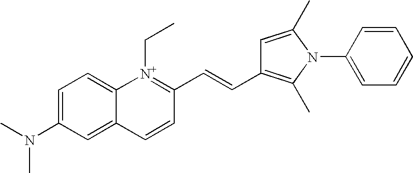

To a solution of 6-dimethylamino-2-methyl-1-ethylquinolinium ethylsulfate (0.120 g, 0.352 mmol) and 2,5-dimethyl-1-phenyl-1H-pyrrole-3-carbaldehyde (70 mg, 0.352 mmol) in MeOH (1 mL) was added piperidine (1-2 drops) and refluxed for 4 h The solution was cooled to room temperature and the precipitate collected using vacuum filtration to afford a red solid (78 mg, 44%). ¹H NMR (DMSO-d₆) δ 8.53-8.47 (m, 2H), 8.23 (d, J=9.8 Hz, 1H), 8.15 (d, J=15.0 Hz, 1H), 7.62-7.58 (m, 3H), 7.56-7.54 (m, 1H), 7.37 (dd, J=3.5, 1.4 Hz, 2H), 7.24 (d, J=3.0 Hz, 1H), 7.16 (d, J=15.1 Hz, 1H), 6.77 (s, 1H), 4.96 (q, J=7.1 Hz, 2H), 3.73 (q, J=7.0 Hz, 2H), 3.11 (s, 6H), 2.23 (s, 3H), 2.01 (s, 3H), 1.50 (t, J=7.1 Hz, 3H), 1.10 (t, J=6.9 Hz, 3H); HPLC Rₜ=12.8

### Example 14

### Synthesis of 6-bromo-2-[(E)-2-(2,5-dimethyl-1-phenyl-1H-pyrrol-3-yl)-vinyl]-1-methyl-quinolinium triflate

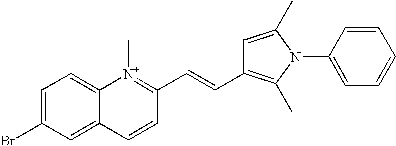

To a solution of 6-bromo-1,2-dimethylquinolinium triflate (0.200 g, 0.517 mmol) and 2,5-dimethyl-1-phenyl-1H-pyrrole-3-carbaldehyde (103 mg, 0.517 mmol) in MeOH (1 mL) was added piperidine (1-2 drops) and refluxed for 4 h The solution was cooled to room temperature and the precipitate collected using vacuum filtration to afford a red solid (99 mg, 34%). ¹H NMR (DMSO-d₆) δ 8.70 (d, J=9.4 Hz, 1H), 8.64 (d, J=9.3 Hz, 1H), 8.49 (d, J=2.3 Hz, 1H), 8.38 (d, J=14.8 Hz, 2H), 8.33 (d, J=9.4 Hz, 1H), 8.18 (dd, J=9.3, 2.3 Hz, 1H), 7.62-7.59 (m, 2H), 7.57-7.54 (m, 1H), 7.39 (dd, J=3.5, 1.4 Hz, 1H), 7.28 (d, J=15.0 Hz, 1H), 6.84 (s, 1H), 4.37 (s, 3H), 2.29 (s, 3H), 2.02 (s, 3H); HPLC Rₜ=11.9.

### Example 15

### Synthesis of 6-dimethylamino-2-[(E)-2-(2,5-dimethyl-1-butyl-1H-pyrrol-3-yl)-vinyl]-1-methyl-quinolinium triflate

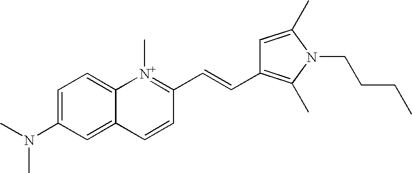

To a solution of A 6-dimethylamino-1,2-dimethylquinolinium triflate (0.200 g, 0.571 mmol) and 1-butyl-2,5-dimethyl-1H-pyrrole-3-carbaldehyde (300 mg, 0.571 mmol) in MeOH (1 mL) was added piperidine (1-2 drops) and refluxed for 4 h The solution was cooled to room temperature and the precipitate collected using vacuum filtration to afford a red solid. The solid was further purified using SiO2 column chromatography using 10% acetone in chloroform (57 mg, 20%). ¹H NMR (DMSO-d₆) δ 8.46 (d, J=9.2 Hz, 1H), 8.41 (d, J=9.4 Hz, 1H), 8.18 (d, J=2.3 Hz, 1H), 8.04 (d, J=15.0 Hz, 1H), 7.57 (dd, J=9.7, 3.1 Hz, 1H), 7.21 (J=3.0 Hz, 1H), 7.08 (d, J=15.0 Hz, 1H), 6.54 (s, 1H), 4.30 (s, 3H), 3.83 (t, J=7.8 Hz, 2H), 3.09 (s, 6H), 2.49 (s, 3H), 2.23 (s, 3H), 1.58-1.55 (m, 2H), 1.36-1.31 (m, 2H), 0.92 (t, J=4.0 Hz, 3H); HPLC Rₜ=12.6

### Example 16

### Synthesis of 6-bromo-2-[(E)-2-(2,5-dimethyl-1-phenyl-1H-pyrrol-3-yl)-vinyl]-1-ethyl-quinolinium triflate

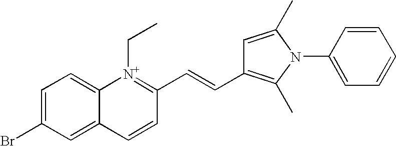

To a solution of A 6-bromo-1-ethyl-2-methylquinolinium triflate (0.200 g, 0.499 mmol) and 2,5-dimethyl-1-phenyl-1H-pyrrole-3-carbaldehyde (100 mg, 0.499 mmol) in MeOH (1 mL) was added piperidine (1-2 drops) and refluxed for 4 h The solution was cooled to room temperature and the precipitate collected using vacuum filtration to afford a red solid (50 mg, 17%). ¹H NMR (DMSO-d₆) δ 8.72 (d, J=11.2 Hz, 1H), 8.64 (d, J=9.3 Hz, 1H), 8.50 (d, J=2.3 Hz, 1H), 8.43 (d, J=14.8, 1H), 8.34 (d, J=9.5 Hz, 1H), 8.18 (dd, J=9.4, 2.3 Hz, 1H), 7.63-7.60 (m, 3H), 7.39-7.38 (m, 2H), 7.20 (d, J=14.9 Hz, 1H), 6.87 (s, 1H), 4.97 (q, J=7.1 Hz, 2H), 2.29 (s, 3H), 2.02 (s, 3H), 1.50 (t, J=7.1 Hz, 3H); HPLC Rₜ=12.9

### Example 17

### Synthesis of 6-Dimethylamino-2-[(E)-2-(2,5-dimethyl-1-phenyl-1H-pyrrol-3-yl)-vinyl]-1-ethyl-quinolinium triflate

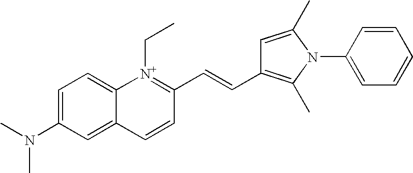

To a solution of A 6-dimethylamino-1-ethyl-2-methylquinolinium triflate (0.125 g, 0.343 mmol) and 2,5-dimethyl-1-phenyl-1H-pyrrole-3-carbaldehyde (68 mg, 0.343 mmol) in MeOH (1 mL) was added piperidine (1-2 drops) and refluxed for 4 h The solution was cooled to room temperature and the precipitate collected using vacuum filtration to afford a red solid (30 mg, 16%). ¹H NMR (DMSO-d₆) δ 8.52-8.47 (m, 2H), 8.23 (d, J=9.9 Hz, 1H), 8.14 (d, J=15.1 Hz, 1H), 7.62-7.52 (m, 4H), 7.37-7.35 (m, 2H), 7.24 (d, J=3.0 Hz, 1H), 7.16 (d, J=15.1 Hz, 1H), 6.77 (s, 1H), 4.96 (q, J=7.5 Hz, 2H), 3.11 (s, 6H), 2.24 (s, 3H), 2.12 (s, 3H), 1.50 (t, J=7.1 Hz, 1H); HPLC Rₜ=12.9.

### Example 18

### Synthesis of 2-[(E)-2-(2,5-dimethyl-1-phenyl-1H-pyrrol-3-yl)-vinyl]-1-methyl-quinolinium triflate

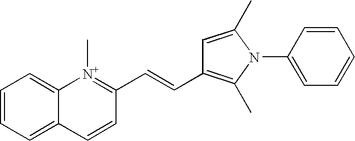

To a solution of 1,2-dimethylquinolinium triflate (0.200 g, 0.651 mmol) and 2,5-dimethyl-1-phenyl-1H-pyrrole-3-carbaldehyde (130 mg, 0.651 mmol) in MeOH (1 mL) was added piperidine (1-2 drops) and refluxed for 4 h The solution was cooled to room temperature and the precipitate collected using vacuum filtration to afford a black solid (113 mg, 36%). ¹H NMR (DMSO-d₆) δ 8.75 (d, J=9.2 Hz, 1H), 8.66 (d, J=9.0 Hz, 1H), 8.40 (d, J=9.0 Hz, 1H), 8.34 (d, J=15.0 Hz, 1H), 8.22 (dd, J=8.0, 1.5 Hz, 1H), 8.08-8.05 (m, 1H), 7.83 (t, J=7.8 Hz, 1H), 7.62-7.54 (m, 3H), 7.39-7.37 (m, 2H), 7.30 (d, J=15.0 Hz, 1H), 6.83 (s, 1H), 4.40 (s, 3H), 2.28 (s, 3H), 2.02 (2, 3H); HPLC Rₜ 11.2.

### Example 19

### Synthesis of 6-bromo-2-[(E)-2-(2,5-dimethyl-1-pyridin-3-yl-1H-pyrrol-3-yl)-vinyl]-1-methyl-quinolinium triflate

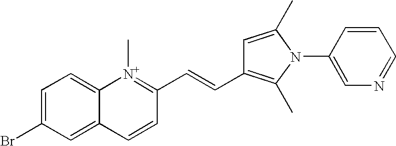

To a solution of 6-bromo-1,2-dimethylquinolinium triflate (0.200 g, 0.518 mmol) and 2,5-dimethyl-1-pyridin-3-yl-1H-pyrrole-3-carbaldehyde (104 mg, 0.518 mmol) in MeOH (1 mL) was added piperidine (1-2 drops) and refluxed for 4 h The solution was cooled to room temperature and the precipitate collected using vacuum filtration to afford a dark purple solid (34 mg, 12%). ¹H NMR (DMSO-d₆) [symbol] 8.76-8.66 (m, 4H), 8.51 (d, J=2.3 Hz, 1H), 8.39 (s, 1H), 8.35 (d, J=10.0 Hz, 1H), 8.20 (dd, J=9.4, 2.4 Hz, 1H), 7.95-7.93 (m, 1H), 7.67-7.65 (m, 1H), 7.32 (d, J=14.9 Hz, 1H), 6.83 (s, 1H), 4.39 (s, 3H), 2.31 (s, 3H), 2.04 (s, 3H); HPLC Rₜ=9.0.

### Example 20

### Synthesis of 6-methyl-2-[(E)-2-(2,5-dimethyl-1-pyridin-3-yl-1H-pyrrol-3-yl)-vinyl]-1-ethyl-quinolinium iodide

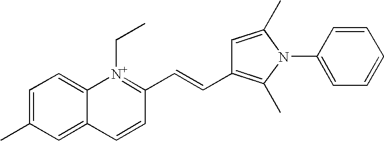

To a solution of 1-ethyl-2,6-dimethylquinolinium iodide (0.025 g, 0.080 mmol) and 2,5-dimethyl-1-phenyl-1H-pyrrole-3-carbaldehyde (16 mg, 0.080 mmol) in MeOH (1 mL) was added piperidine (1-2 drops) and refluxed for 4 h The solution was cooled to room temperature and the precipitate collected using vacuum filtration to afford a color solid (5 mg, 13%). HPLC Rₜ=12.0

### Example 21

### Synthesis of 6-chloro-2-[(E)-2-(2,5-dimethyl-1-phenyl-1H-pyrrol-3-yl)-vinyl]-1-methyl-quinolinium triflate

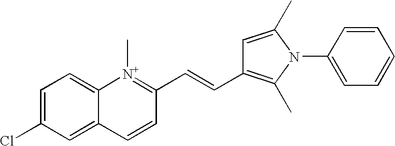

To a solution of 6-chloro-1,2-dimethylquinolinium triflate (0.200 g, 0.585 mmol) and 2,5-dimethyl-1-phenyl-1H-pyrrole-3-carbaldehyde (117 mg, 0.585 mmol) in MeOH (1 mL) was added piperidine (1-2 drops) and refluxed for 4 h The solution was cooled to room temperature and the precipitate collected using vacuum filtration to afford a solid (85 mg, 28%). ¹H NMR (DMSO-d₆) δ 8.71 (d, J=9.5 Hz, 1H), 8.65 (d, J=9.3 Hz, 1H), 8.40 (m, J=9.4 Hz, 2H), 8.36-8.35 (m, 1H), 8.08 (dd, J=9.4, 2.5, 1H), 7.63-7.54 (m, 3H), 7.40-7.38 (m, 2H), 7.28 (d, J=14.9 Hz, 1H), 6.84 (s, 1H), 4.38 (s, 3H), 2.29 (s, 3H), 2.01 (s, 3H); HPLC Rₜ=11.7.

### Example 22

### Synthesis of 7-chloro-2-[(E)-2-(2,5-dimethyl-1-phenyl-1H-pyrrol-3-yl)-vinyl]-1-methyl-quinolinium triflate

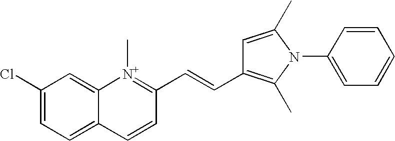

To a solution of 7-chloro-1,2-dimethylquinolinium triflate (0.200 g, 0.585 mmol) and 2,5-dimethyl-1-phenyl-1H-pyrrole-3-carbaldehyde (117 mg, 0.585 mmol) in MeOH (1 mL) was added piperidine (1-2 drops) and refluxed for 4 h The solution was cooled to room temperature and the precipitate collected using vacuum filtration to afford a solid (99 mg, 32%). ¹H NMR (DMSO-d₆) δ 8.73. (d, J=9.3 Hz, 1H), 8.67 (d, J=9.4 Hz, 1H), 8.53 (d, J=1.6 Hz, 1H), 8.38 (d, J=14.9 Hz, 1H), 8.23 (d, J=8.6 Hz, 1H), 7.87 (dd, J=8.3, 1.7 Hz, 1H), 7.63-7.53 (m, 3H), 7.40-7.38 (m, 2H), 7.28 (d, J=14.9 Hz, 1H), 6.86 (s, 1H), 4.37 (s, 3H), 2.29 (s, 3H), 2.01 (s, 3H); HPLC Rₜ=11.8

### Example 23

### Synthesis of 6-methoxy-2-[(E)-2-(2,5-dimethyl-1-phenyl-1H-pyrrol-3-yl)-vinyl]-1-methyl-quinolinium triflate

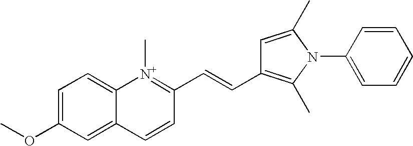

To a solution of 6-methoxy-1,2-dimethylquinolinium triflate (0.200 g, 0.593 mmol) and 2,5-dimethyl-1-phenyl-1H-pyrrole-3-carbaldehyde (118 mg, 0.593 mmol) in MeOH (1 mL) was added piperidine (1-2 drops) and refluxed for 4 h The solution was cooled to room temperature and the precipitate collected using vacuum filtration to afford a purple solid (114 mg, 37%). ¹H NMR (DMSO-d₆) δ 8.66 (d, J=9.2 Hz, 1H), 8.61 (d, J=9.3 Hz, 1H), 8.35 (d, J=9.5 Hz, 1H), 8.23 (d, J=15.0 Hz, 1H), 7.72-7.68 (m, 2H), 7.62-7.53 (m, 3H), 7.38 (m, 2H), 7.27 (d, J=15.0 Hz, 1H), 6.78 (s, 1H), 4.40 (s, 3H), 3.03 (s, 3H), 2.26 (s, 3H), 2.01 (s, 3H); HPLC Rₜ=11.5

### Example 24

### Synthesis of: 2-(2-(4-benzyloxy-3-chlorophenyl)vinyl)-6-dimethylamino-1-methylquinolinium methanesulfonate

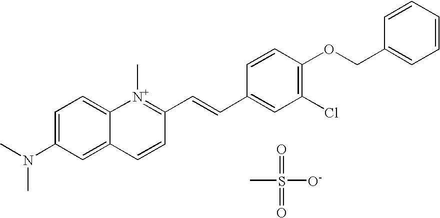

A sample of 6-(dimethylamino)-1,2-dimethylquinolinium methanesulfonate, 120 mg, and 4-benzyloxy-3-chlorobenzaldehyde, 180 mg, in 1 ml of butanol was treated with 10 ul of piperidine, and warmed to 100° C. for 1 hour. The mix was cooled to −20° C. over night, precipitating solids. On warming to room temperature, the solids dissolved. The solution was treated with 2 ml of ethyl acetate, and then cooled to −20° C. The resulting solids were collected by filtration, washed with 2 ml ethyl acetate and air dried to give 68 mg of red solids, HPLC method 0100CD Rₜ 12.118 min, 99% pure.

NMR D6DMSO 500 MH 8.70 J=9 D 1H, 8.34 J=9.9 D 1H, 8.29 J=9.15 D 1H, 8.18 J=2.1 D 1H, 7.94 J=15.9 D 1H, 7.81 J=8.7, 2.2 DD 1H, 7.79 J=16 D 1H, 7.73 J=9.8, 3.0 DD 1H, 7.50 J=7.4 D 2H, 7.45-7.35 M 4H, 7.26 J=3 D, 1H, 5.33 S 2H, 4.49 S 3H, 3.14 S 6H, 2.28 S 3H.

### Examples 25 to 58

The following examples were prepared following the method of Example 23:

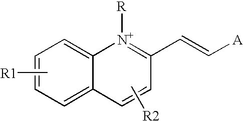

TABLE 3
Examples 25 to 58
R2 = H, as triflate salt

| | | | | EC50 |
|---|---|---|---|---|
| Example | R1 | R | A | uM |
| 25 | 6-Me2N- | Me | 4-formyl-2,5-dimethyl-1-(2- | <1 |
| | | | methoxyethyl)-pyrrol-3-yl | |
| 26 | 6-(4-morpholinyl) | Me | 2,5-dimethyl-1-phenylpyrrol-3-yl | 1 |
| 27 | 6-(4-morpholinyl) | Me | 2,5-dimethyl-1-butyl-4-formylpyrrol- | >1 |
| | | | 3-yl | |
| 28 | 6-Me2N- | Me | 2,5-dimethyl-1-phenyl-4- | <1 |
| | | | formylpyrrol-3-yl | |
| 29 | 6-(Piperidine-1-yl) | Me | 2,5-dimethyl-1-phenylpyrrol-3-yl | 1 |
| 30 | 6-(4- | Me | 2,5-dimethyl-1-phenylpyrrol-3-yl | 1 |
| | methylpiperidin-1- | | | |
| | yl) | | | |
| 31 | 6-Me2N- | Me | 6-(thiophen-2-yl)pyridine-2-yl | 1 |
| 32 | 6-Me2N- | Me | 5-phenylthiophen-2-yl | 1 |
| 33 | 6-Me2N- | Me | 4,5-dimethylfuran-2-yl | 1 |
| 34 | 6-Me2N- | Me | 3-phenoxyphenyl | 1 |
| 35 | 6-Me2N- | Me | 4-phenoxyphenyl | <1 |
| 36 | 6-Me2N- | Me | 4-butyloxyphenyl | <1 |
| 37 | 6-Me2N- | Me | 5-(2-methyl-5-trifluoromethyl-2H- | 1 |
| | | | pyrazol-3-yl)thiophen-2-yl | |
| 38 | 6-Me2N- | Me | 5-(1-methyl-4-trifluoromethyl-1H- | 1 |
| | | | pyrazol-3-yl)thiophen-2-yl | |
| 39 | 6-Me2N- | Me | 2-butyl-5-chloro-imidazol-4-yl | >1 |
| 40 | 6-Me2N- | Me | Chromene-4-one-3-yl | 1 |
| 41 | 6-Me2N- | Me | 1-phenylsulfonyl-indol-3-yl | 1 |
| 42 | 6-Me2N- | Me | 2-(4-chlorophenyl)carbonyl- | 1 |
| | | | benzofuran-5-yl | |
| 43 | 6-Me2N- | Me | 4-(2-cyanothiophen-3-yl- | <1 |
| | | | methyloxy)phenyl | |
| 44 | 6-Me2N- | Me | 4-trifluoromethoxy-phenyl | 1 |
| 45 | 6-Me2N- | Me | 4-benzyloxy-3-methoxy-phenyl | <1 |
| 46 | 6-Me2N- | Me | 4-benzyloxyphenyl | <1 |
| 47 | 6-Me2N- | Me | 4-benzyloxy-3,5-dimethylphenyl | <1 |
| 48 | 6-Me2N- | Me | 5-methyl-1-phenylpyrazol-4-yl | >1 |
| 49 | 6-Me2N- | Me | 3-phenoxythiophen-2-yl | <1 |
| 50 | 6-Me2N- | Me | 5-(2-phenylacetlyene)-thiophen-2-yl | <1 |
| 51 | 6-Me2N- | Me | 9-ethylcarbazol-3-yl | <1 |
| 52 | 6-Me2N- | Me | 4-(4-flourobenzoyl)-1-methylpyrrol-2-yl | IA |

The following compounds are methanesulfonate

| | | | | |
|---|---|---|---|---|
| 53 | 6-Me2N- | Me | 1-cyclopropyl-2,5-dimethyl-4- | <1 |
| | | | ethoxycarbonylpyrrol-3-yl | |
| 54 | 6-Me2N- | Me | 2,5-dimethyl-1-phenyl-4- | <1 |
| | | | ethoxycarbonylpyrrol-3-yl | |
| 55 | 6-Me2N- | Me | 2,5-dimethyl-1-(thiophen-2- | <1 |
| | | | yl)methyl-4-ethoxycarbonylpyrrol-3-yl | |
| 56 | 6-Me2N- | Me | 3-formyl-2,5-dimethyl-1-butylpyrrol- | <1 |
| | | | 4-yl | |
| 57 | 6-Me | Me | 3-formyl-2,5-dimethyl-1-butylpyrrol- | <1 |
| | | | 4-yl | |
| 58 | 6-Cl | Me | 3-formyl-2,5-dimethyl-1-butylpyrrol-4-yl | <1 |

### Example 59

### Synthesis of 3-formyl-2,5-dimethyl-1-(2-phenylethyl)pyrrol-4-yl

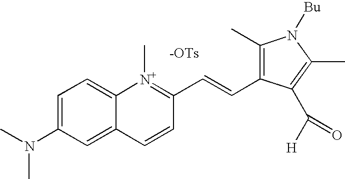

To a solution of 1,2-dimethylquinaldine (4.08 g, 10.9 mmol) and 2,5-dimethylpyrrole (2.25 g) in 4 mL of ethanol was added one drop of piperidine, which was then heated to reflux for 16 hours. The solution was cooled and the precipitate filtered to give 1.8 grams of impure product. The impure product was recrystallized using anhydrous EtOH (60% recovery).

¹H NMR (DMSO-d₆) δ 10.00 (s, 1H), 8.59 (d, J=9.2, 1H), 8.41 (d, J=9.2, 1H), 8.34 (d, J=15.0, 1H), 8.25 (d, J=9.8, 1H), 8.00 (d, J=15.4, 1H), 7.67-7.65 (m, 1H), 7.46 (dd, J=6.4, 1.8, 2H), 7.25 (d, J=3.0, 1H), 7.11 (d, J=8, 2H), 4.41 (s, 3H), 3.99-3.97 (m, 2H), 3.12 (s, 6H), 2.59 (s, 3H), 2.53 (s, 3H), 2.28 (s, 3H), 1.62-1.59 (m, 2H), 1.39-1.43 (m, 2H), 0.95 (t, J=7.3, 1H).

### Example 60

### Reduction in Anchorage-Dependent Growth (Normal Liquid Cell Culture) of Cancer Cells

The compound of Example 1 was tested in liquid culture with and without added glucose in 96 well plates to ascertain its effectiveness in reducing cancer cell growth. The method is as follows:

Cells were cultured in 1× Dulbecco's modified Eagle's medium (DMEM) supplemented with 10% fetal bovine serum (FBS), 2 mM L-Glutamine (L-Glu), 1× Nonessential Amino Acids (NEAAs), 1% sodium pyruvate, with or without 4.5 g/L of glucose, all from Invitrogen, Carlsbad, Calif., USA) in a humidified incubator (Ultra-Tech WJ301D; Baxter Scientific Products, West Chester, Pa., USA) with 5% CO2 at 37° C. Cell suspensions (100 ml) containing 1-3×10³ cells were plated into each well of a 96-well flat-bottom microplate. The cells were allowed to grow for 1-3 days before the cell growth was measured using alamarBlue™ staining (1:10 volume reagent; Biosource International, Camarillo, Calif., USA), according to the manufacturer's instructions.

TABLE 4
Selective potency of the compound of Example 1 against cancer
cells in liquid growth cultures with and without added glucose
(indicated with a (+) Glu and (−) Glu).

| | (−) Glu | (+) Glu | (−) Glu | (+) Glu | (−) Glu | (+) Glu |
|---|---|---|---|---|---|---|
| Cell line | Panc-1 | Panc-1 | HCT-116 | HCT-116 | Pc3M/N | Pc3M/N |
| IC80 uM | 0.1 | >1 | 0.1 | 1 | 0.1 | 1 |

The compounds of Examples 25 through 59 were tested as indicated and provided at least a 50% reduction of viable cell growth at the concentration stated in Table 3, using glucose deprived media and Panc-1, HCT-116, Pc3M/N, HEPG2, ASPC1, or H460 cells, except for compound 52, which was inactive.

### Example 61

### Reduction in Anchorage-Dependent Growth (Soft Agar) of Cancer Cells

The compound of Example 1 was further tested in both liquid culture and soft agar. More specifically, cells were cultured in either 1× Dubecco's modified Eagle's medium (DMEM), supplemented with 10% fetal bovine serum (FBS), 2 mM L-Glutamine, 1× Non-Essential Amino Acids, and 1% sodium pyruvate, or RPMI 1640 supplemented with 10% FBS and 2 mM L-Glutamine, all from Invitrogen, Carlsbad, Calif., USA in a humidified incubator (Ultra-Tech WJ301D; Baxter Scientific Products, West Chester, Pa., USA) with 5% CO₂ at 37° C. Cell suspensions containing 1×10³ cells were plated into each well of a 96-well flat-bottom microplate in liquid culture medium or semi-solid agar culture media and incubated at 37° C. for 3 days or 7 days respectively. Cell growth was measured by alamarBlue™ staining (1:10 volume reagent; Biosource International, Camarillo, Calif., USA) using a PerSeptive Biosystems CytoFluor® Series 4000 Multi-Well Plate Reader, with excitation at 530±25 nm and emission at 590±35 nm. The results are shown in Table 5 below.

TABLE 5
Potency of the compound of Example 1 against cancer cells in liquid
culture and soft agar culture.

| | | IC50 (uM) | |
|---|---|---|---|
| Cell type | Cell line | Liquid culture | Soft agar |
| Breast cancer | MCF7 | 0.03-0.1 | <0.03 |
| | T47D | ~0.03 | <0.03 |
| | SKBR3 | ~1 | ~1 |
| | MDA-231 | 0.1-0.3 | <0.03 |
| | MDA_MB-435 | 0.3-1 | 0.03-0.1 |
| | HS578 | 0.1-0.3 | 0.03-0.1 |
| Colon cancer | HCT116 | >1 | 0.1-0.3 |
| | HCT116-P53(−) | >1 | 0.1-0.3 |
| | HT29 | >1 | 0.03-0.1 |
| | SW480 | 0.3-1 | 0.3-1 |
| | DLD1 | 0.3-1 | ~1 |
| Prostate cancer | PC3M/N | >1 | ~0.3 |
| | PC3 | 0.1-0.3 | 0.1-0.3 |
| | DU145 | >1 | 0.3-1 |
| Pancreatic cancer | Panc-1 | 0.3-1 | |
| | ASPC-1 | 0.3-1 | |
| Ovarian C. cancer | A2780 | 0.03-0.1 | 0.03-0.1 |
| | OVCAR-1 | 0.03-0.1 | |
| | PA-1 | 0.03-0.1 | |
| | OVCAR-8 | 0.1-0.3 | 0.03-0.1 |
| | SK-OV-3 | 0.1-0.3 | 0.03-0.1 |
| | OVCAR-4 | 0.1-0.3 | |
| | OVCAR-5 | | 0.03-0.1 |
| | Caov-3 | 0.03-0.1 | |
| NSCL cancer | A549 | 0.1-0.3 | 0.033-0.1 |
| | NCI-H460 | 0.1-0.33 | 0.03-0.1 |
| Liver cancer | Hep3B | 0.3-1 | |
| Melanoma | UACC62 | 0.1-0.3 | 0.03-0.1 |
| | UACC257 | ~0.1 | 0.1-0.3 |
| | A2058 | 0.1-0.3 | |
| Normal | HUVEC | 0.03-0.1 | |
| | W138 | 0.1-0.3 | |
| | IMR90 | 0.1-0.3 | |
| | CCD112/N | ~1 | |

As shown in Table 5, the compound of Example 1 (at the concentration level listed) showed a 50% reduction of cell growth of the cell lines listed in either liquid or soft agar culture.

### Example 62

### Reduction of Tumor Size in Colon Cancer Xenograft Model

The anti-tumor activity of the compound of Example 1 was tested in a nude mice xenograft advanced stage model utilizing two different forms of monotherapy: i.p. and oral dosings. Female athymic nude (nu/nu) mice were inoculated with HCT116 colon cancer cells, and the tumors were allowed to grow to a volume of about 150 mm³. The mice were then dosed with the compound of Example 1 or a control, either orally or i.p. The results, as depicted in FIG. 1, show a significant reduction in tumor volume, as compared to control, with no apparent toxicity, i.e., minimal change in body weight (the compound of Example 1 is called IMST8-p in FIG. 1).

### Example 63

### Reduction of Tumor Growth in Non-Small Cell Lung Cancer (NSCL) Xenograft Model

The anti-tumor activity of the compound of Example 56 was tested in a nude mice xenograft early stage model. Female athymic nude (nu/nu) mice were inoculated with H460 NSCL cells and divided into two groups. The first group was dosed with vehicle (control), and the second group was dosed i. p. with the compound of Example 56 on a daily basis (6 times/week). As shown in FIG. 2, a significant reduction of H460 tumor size (p<0.05) was observed as compared to control in the mice which received the compound of Example 56 (labeled IMST8D1b in FIG. 2) with no apparent toxicity.

### Example 64

### Synergistic Combination of Taxol and the Compound of Example 1

The anti-tumor activity of compounds of the invention in combination with at least one additional chemotherapeutic agent was also tested in a nude mice xenograft model.

A. Female athymic nude (nu/nu) mice were inoculated with PC3 prostate cells. When tumor volumes reached around 100 mm³, the animals were divided into four groups with average size of tumors similar among the four groups. The first group was dosed with vehicle (as control), the second group with the compound of Example 1 (P.O. daily), the third with doxorubicin (i.p.) and the fourth with the combination (i.p. Dox/p.o. compound of Example 1). The results are shown in FIG. 3 (the compound of Example 1 is called IMST8 in FIG. 3). As can be seen from FIG. 3, the combination therapy of the compound of Example 1 and doxorubicin was very effective in reducing the growth of PC3 medium-staged xenograft tumors.

B. Female athymic nude (nu/nu) mice were inoculated with PC3 prostate cells. When tumor volumes reached around 100 mm³, the animals were divided into four groups with average size of tumors similar among the four groups. The first group was dosed with vehicle (as control), the second group with the compound of Example 1 (P.O. daily), the third with paclitaxel (taxol) (i.p.) and the fourth with the combination (ip. taxol/p.o. compound of Example 1). The results are shown in FIG. 4 (the compound of Example 1 is called IMST8 in FIG. 4). As can be seen from FIG. 4, the paclitaxel (taxol) monotherapy reduced tumor size significantly; and the combination therapy of the compound of Example 1 and paclitaxel demonstrated a further reduction in tumor growth.

Table 6 below summarizes the results of the xenograft experiments showing the efficacy of the compounds of the invention (i.e., the compound of Example 1 or Example 56) in reducing or inhibiting tumor growth.

TABLE 6

| Tumor | | | |
|---|---|---|---|
| type | Cells | Dosing regimens | Result |
| Colon | HCT116 | Example 1 (I.p.) | Tumor reduction (25%) |
| Prostate | PC3 | Example 1 | Tumor reduction (72%) |
| | | (+doxorubicin) | |
| Prostate | PC3 | Example 1 (+pacitaxel) | Tumor reduction (83%) |
| NSCL | H460 | Example 56 (I.p.) | Tumor reduction (41%) |

### Example 65

### Synergistic Combination of Taxol and the Compound of Example 47

Combination treatment with taxol and the compound of Example 47 (also designated IMS2155) in the A549 (NCLC) model was tested. Taxol is one of standards of care for lung cancer.

Female athymic nude mice (nu/nu) (4-6 weeks old) were inoculated (s.c.) with 5×10⁶ A549 cancer cells in PBS on the flanking site. The animals were divided into four treatment groups when the average tumor size reached 150 mm³: group 1 (PBS daily i.p. dosing), group 2 (20 mg/kg paclitaxel once per 4 days i.p. dosing), group 3 (2 mg/kg IMS2155 daily i.p. dosing) and group 4 (20 mg/kg paclitaxel once per 4 days i.p. dosing and 2 mg/kg IMS2155 daily i.p. dosing). The tumor size and body weight was measured twice per week. Tumor volume was calculated using the formula TV=length×width×width/2.

The responses of the treated tumors are shown in FIG. 5. A significant reduction in tumor size was observed from treatment either with Taxol alone or with IMS2155 alone (p<0.05). The combination of both Taxol and IMS2155 showed an enhanced response, which demonstrated the synergy of the combination. It is noteworthy that IMS2155 worked almost as well as Taxol at the MTD dose (20 mg/kg for this strain of mice).

### Example 66

### Treating Lung Cancer with the Compounds of Examples 36 and 47

Using the mouse Lewis lung cancer (LLC) xenograft model, female C57BL mice (4-6 weeks old) were inoculated (s.c.) with 5×10⁵ Lewis lung cancer cells in PBS on the flanking site. Animals were randomly divided into treatment and control groups (n=8) based on tumor size when average tumor size reached 100 mm³. Animals in the treatment group received the compound of Example 36 (also designated IMS2143) or the compound of Example 47 daily at 2 mg/kg via intraperitoneal dosing, 6 times per week respectively. The control group was injected with 0.9% NaCl. Tumor size and body weight were measured twice per week. Tumor volume was calculated using the formula TV=length×width×width/2.

As shown in FIG. 6, the compounds of Examples 36 and 47 each significantly suppressed tumor growth at day 15 (p<0.05).

### Example 67

### Synergistic Anti-Tumor Effect of the Compound of Example 36 with 2DG In Vivo

2-deoxyglucose (2DG) is a glucose analog and glycolysis inhibitor. The fluorescence labeled analog fluorodexoxyglucose F18 (FDG) has broadly been used as a tumor imaging agent for measuring glucose uptake by tumors in contrast to normal tissues. As a glycolysis inhibitor, it causes transcriptional activation of GRP78 or UPR response, similar to that seen in glucose starvation. Therefore, 2DG is a useful hypoglycemia-mimicking agent. 2DG is also currently under evaluation as a potential therapeutic for cancers in combination with other chemotherapy such as adriamycin and Taxol.

Because the compounds of the invention may be more toxic to cancer cells deprived of glucose or treated with 2DG in vitro, the combination of 2DG with such compounds should have increased anti-tumor efficacy in vivo as compared with either agent alone. Accordingly, the effect of a combination therapy of the compound of Example 36 and 2DG using A549 NSCL xenograft model was examined.

Female athymic nude mice (nu/nu) (4-6 weeks old) were inoculated (s.c.) with 5×10⁶ A549 cancer cells in PBS on the flanking site. After the tumors reached an average size ˜100 mm³, the animals were divided into four treatment groups—0.9% NaCl (control), 2DG at 300 mg/kg, the compound of Example 36 at 2 mg/kg, and a combination of 2DG (300 mg/kg) and the compound of Example 36 (2 mg/kg). All the treatments were delivered by intraperitoneal injection daily (6 times per week). Tumor size and body weight were measured twice per week. Tumor volume was calculated using the formula TV=length×width×width/2.

As shown in FIG. 7, no response was observed for 2DG monotherapy. However, monotherapy with the compound of Example 36 significantly inhibited tumor growth at day 24 and day 27 (p<0.05) compared to both control and 2DG alone group. The combination of the compound of Example 36 and 2DG showed increased efficacy (p<0.05 at day 13, 17, 20, 24 and 27, as compared to both control and 2DG alone).

The synergistic effect of the combination of the compound of Example 36 with 2DG was further demonstrated in a mouse LLC model. More specifically, female C57BL mice (4-6 weeks old) were inoculated (s.c.) with 5×10⁵ Lewis lung cancer cells in PBS on the flanking site. Animals were randomly divided into treatment and control groups (n=8) based on tumor size when average tumor size reached 100 mm³. Animals in the treatment groups received the compound of Example 36 daily at 2 mg/kg and 2DG at 300 mg/kg via intraperitoneal dosing, 6 times per week respectively. Tumor size and body weight were measured twice per week. Tumor volume was calculated using the formula TV=length×width×width/2.

As shown in FIG. 8, both the compound of Example 36 alone and in combination with 2DG significantly inhibited tumor growth as compared with 2DG alone or control group (p<0.05).

All references cited herein are incorporated in their entirety. It is appreciated that the detailed description above is intended only to illustrate certain preferred embodiments of the present invention. It is in no way intended to limit the scope of the invention, as set out in the claims.

### REFERENCES

1 U.S. Pat. No. 2,298,731 and U.S. Pat. No. 2,409,612
2 U.S. Pat. No. 3,615,608
3 U.S. Pat. No. 2,965,485
4 U.S. Pat. No. 2,515,912.
5 Wizinger and Wenning H., Über intramolekulare Ionisation; Helv. Chim. Acta, 23:247-271(1940), 10.1002/hlca.19400230133
6 Lugowkin, J. Gen. Chem. USSR (Engl. Transl.), 32:445, (1962); Chem. Abstr.; 58:521 (1963).
7 Lugowkin, J. Gen. Chem. USSR, (Engl. Transl.), 33:2868 (1963).
8 Zhmurova, et al., JGCHA4; J. Gen. Chem. USSR, (Engl. Transl.), EN, 43:1772-1776 (1973).
9 Pilyugin et al., J. Gen. Chem. USSR, (Engl. Transl.), 44:379 (1974).
Stepanov and Lukashina, J. Gen. Chem. USSR (Engl. Transl.), 33:2303-2306 (1963).
11 Chernyuk et al., J. Gen. Chem. USSR (Engl. Transl.), 45: 1525 (1975).
12 Buttgereit, Ber. Bunsen-Ges. Phys. Chem., 69:301 (1965).
13 Mal'tseva, Stepanov, J. Org. Chem. USSR (Engl. Transl.), 9:1792 (1973).
14 Rout, M. K., J. Proc. Inst. Chem.(India), 35:117, 128 (1963); Chem. Abstr., 59:15412.
15 Boyer, J. Chim. Phys. Phys. Chim. Biol, 57:381, 382 (1960).
16 Osman et al., J. Appl. Chem. Biotechnol., 26: 762, 763, 766 (1976).
17 Wainright and Kristiansen, Int. Jour. Antimicrobial Agents, 22:479-486 (2003).
18 Esumi et al., Cancer Sci., 95:685-90 (2004).
19 U.S. Pat. No. 2,925,417.
20 IUPAC Handbook of Pharmaceutical Salts, Properties, Selection and Use, P. Heinrich Stahl, Camille G. Wermuth, Wiley-VCH
21 Chem Ab, 101:166571
22 U.S. Pat. No. 2,715,622; Chen Ab, 50:p5246g
23 Heslot et al., Eff Ioniz Radiat Seeds Proc Symp (1960), 243-249 (1961)
24 http://wwwdotoehhadotorg/prop65/out_of_date/598notdothtml
25 U.S. App. Pub. No. 2004097408
26 Ciufolini, M. A., Tet Lett, 37:8281 (1996).
27 http://wwwdotchdoticdotacdotuk/montalban/PhDdotpdf

1-24. (canceled)

25. A compound of the formula: 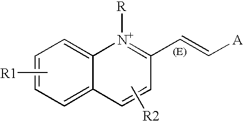
wherein:
A is selected from the group consisting of phenyl, substituted phenyl, heteroaryl and substituted heteroaryl, where the substitutions can also include —OR₃, —CHO, —CN, —CONR₃R₄ or CO₂R₃;
R is selected from the group consisting of hydrogen, C₁ to C₁₂ alkyl, C₁ to C₁₂ substituted alkyl, phenyl, substituted phenyl, C₇ to C₁₈ phenylalkyl and C₇ to C₁₈ substituted phenylalkyl;
R₁ and R₂ are, independently, selected from the group consisting of hydrogen, C₁ to C₁₂ alkyl, C₁ to C₁₂ substituted alkyl, heterocycle, substituted heterocycle, cyclic C₂ to C₇ heteroalkylene, substituted cyclic C₂ to C₇ heteroalkylene, bicyclic heterocycle, substituted bicyclic heterocycle, halo, —CHO and cyano, or one of the following formulas: —OR₃, —NR₃R₄, —C(O)NR₃R₄ and —CO₂R₃; and
R₃ and R₄ are, independently, selected from the group consisting of hydrogen, C₁ to C₁₂ alkyl, C₂ to C₁₂ alkenyl, C₂ to C₁₂ alkynyl, C₁ to C₁₂ substituted alkyl, C₂ to C₁₂ substituted alkenyl, C₂ to C₁₂ substituted alkynyl, C₇ to C₁₈ phenylalkyl and C₇ to C₁₈ substituted phenylalkyl;
or a pharmaceutically acceptable salt thereof;
provided that at least one of the following exclusions takes place:
a) R is not C₁ to C₄ alkyl or hydroxyethyl;
b) R₁ is not methyl, dimethylamino, methoxy or heptylamino at the 7-position of the depicted quinolyl ring; or
c) A is not a substituted pyrrole, where either i) the substitution at the 2- and 5-positions of the pyrrole is methyl; or ii) the substitution at the 1-position of the pyrrole is phenyl, alkyl, cyclohexyl or methoxyethyl.

26. The compound of claim 25, wherein at least one of the following exclusions takes place:
a) R is not C₁ to C₆ alkyl or hydroxyalkyl; or
b) R₁ is not C₁ to C₄ alkyl, alkoxy, dialkylamino or alkylamino; or
c) A is not a substituted pyrrole, where either i) the substitution at the 2- and 5-positions of the pyrrole is C₁ to C₄ alkyl; or ii) the substitution at the 1-position of the pyrrole is phenyl, benzyl, alkyl, cyclohexyl or alkoxyethyl.

27. The compound of claim 25, wherein, when A is substituted, it is with phenyl, substituted phenyl, heteroaryl or substituted heteroaryl.

28. The compound of claim 27, wherein said phenyl or heteroaryl are substituted with C₁ to C₁₂ alkyl, C₁ to C₁₂ substituted alkyl, OR₃, halo, —CHO, —CN, —CONR₃R₄ or —CO₂R₃.

29. The compound of claim 25, wherein A is an optionally substituted phenyl, thiophenyl, furanyl, indolyl, imidazolyl or isoxazolyl.

30. The compound of claim 29, wherein A is substituted and the substitutions are selected from the group consisting of C₁ to C₁₂ alkyl, C₁ to C₁₂ substituted alkyl, phenyl, phenoxy, benzyloxy, substituted phenyl, —CHO and alkoxy.

31. The compound of claim 30, wherein the substitutions are selected from the group consisting of methyl, butyloxy, butyl, phenyl, phenoxy, benzyl, benzyloxy, —CHO and methoxy.

32. The compound of claim 29, wherein R is alkyl.

33. The compound of claim 32, wherein R is methyl.

33-64. (canceled)

65. The compound of claim 31, wherein A is a substituted phenyl.

66. The compound of claim 65, wherein the substitutions are selected from the group consisting of methyl, butyloxy and benzyloxy.

67. The compound of claim 25, wherein R₁ is —NR₃R₄.

68. The compound of claim 67, wherein R₃ and R₄ are each methyl.

69. The compound of claim 25, wherein R₂ is hydrogen.

70. The compound of claim 25, wherein R₁ is selected from the group consisting of a dialkylamino, halo, C₁ to C₁₂ alkoxy and C₁ to C₁₂ alkyl.

71. The compound of claim 70, wherein R₁ is selected from the group consisting of dimethylamino, bromo, chloro, methoxy and methyl.

72. The compound of claim 70, wherein R₁ is at the 6- or 7-position of the quinolyl.

73. The compound of claim 72, wherein R₁ is at the 6-position of the quinolyl.

74. The compound of claim 72, wherein R₁ is at the 7-position of the quinolyl.
